(19) United States
(12) Patent Application Publication
(10) Pub. No.: US 2012/0189700 A1
(43) Pub. Date: Jul. 26, 2012

(54) Nanoparticle Based Immunological Stimulation

(76) Inventors: Zoraida Aguilar, Fayetteville, AR (US); Yongqiang Wang, Springdale, AR (US); Hengyi Xu, Springdale, AR (US); George Hui, Honolulu, HI (US); Kae Pusic, Honolulu, HI (US)

(21) Appl. No.: 13/350,849

(22) Filed: Jan. 16, 2012

Related U.S. Application Data
(60) Provisional application No. 61/434,073, filed on Jan. 19, 2011.

(51) Int. Cl. A61K 39/385; A61P 35/00; A61K 9/51; B82Y 5/00

(52) U.S. Cl. 424/489; 424/193.1; 977/773; 977/774; 977/810; 977/811; 977/906; 977/917

(57) ABSTRACT

A nanoparticle-based delivery system and methods for its use are disclosed. In one aspect, a nanoparticle-based delivery system comprising at least one molecule such as proteins, DNA/RNA or fragments thereof, carbohydrates, enzymes, chemicals, virus cells, bacteria, parts of a virus, parts of a bacteria, parts of a cell, part of a tissue, or a combination of one or more of these, which shall be referred to as immunogens, are chemically or physically combined with water soluble nanoparticles which, when administered to a living system, is capable of eliciting a desired immunological response. More particularly, the invention relates to nanoparticle-based delivery systems that are specifically engineered to enhance humoral or cellular immune response without the use of adjuvants.

### CROSS REFERENCE TO RELATED APPLICATIONS

This application claims priority to U.S. Provisional Patent Application Ser. No. 60/532,028, entitled NANOPARTICLE-BASED DELIVERY SYSTEMS filed on Jan. 19, 2011, the entirety of which is hereby incorporated by reference.

### STATEMENT REGARDING FEDERALLY SPONSORED RESEARCH AND DEVELOPMENT

The subject matter described herein was funded in part with United States government support under Grant Nos. A1076955 by the National Institutes of Health and Grant No. 1047352 by the National Science Foundation. The government has certain rights to the claimed subject matter

### FIELD OF THE INVENTION

This disclosure generally relates to nanoparticle-based delivery systems suitable for use in biological systems and comprising at least one molecule that is chemically or physically combined with a nanoparticle which, when administered to a biological system, is capable of eliciting a desired biological response. More particularly, the invention relates to nanoparticle-based delivery systems that are specifically engineered to achieve an enhanced immune response.

### BACKGROUND OF THE INVENTION

The immune system of an organism consists of biological structures and processes that protect against disease by identifying and killing pathogens. The immune system accomplishes this by detecting a wide variety of pathogens, from viruses to large parasitic worms to tumor cells, and then initiating a protective response that includes the activation of certain cells (e.g., macrophages, T-cells) and the release of various chemical components (e.g., cytokines, chemokines) to fight the pathogen.

What we call the immune system is actually multiple biological mechanisms that evolved to recognize and neutralize pathogens. The immune system consists of many types of proteins, cells, organs, and tissues that interact in an elaborate and dynamic network that, over time, adapts to recognize specific pathogens more efficiently. This adaptation creates immunological memory from a primary response to a specific pathogen which provides an enhanced response to secondary encounters with the same, specific pathogen. This process is generally referred to as “acquired immunity” and is the basis of vaccination.

One obstacle in developing vaccines is that some antigens (i.e., pieces of virus or bacteria) do not produce an effective immune response when injected directly into a patient. These antigens are often ignored by the antigen-presenting cells (APC's) that initiate portions of an immune response and are cleared rapidly from the system.

In many instances, vaccine efficacy is enhanced by administration of an antigen in combination with an adjuvant. Adjuvants are materials that aid the cellular or humoral immune response to an antigen. Generally speaking, adjuvants aid an immune response by increasing inflammation at the site of vaccine administration (e.g., injection) or stablizing the antigen or creating other conditions to increase the likelihood that the immune system will recognize the antigen and mount a response to it.

Currently, there are limited numbers of adjuvant formulations approved for clinical use, for example MF59, alum, Montanide ISA51, and ASO2A. The development of new adjuvants has not kept up with the increasing demand for their use in vaccine formulations. In addition, adjuvants often influence the quality of the immune responses, which indicates that there is not a single adjuvant formulation that is universally effective for all vaccines.

Vaccines based on recombinant peptide technology are exemplary of the difficulties often encountered in producing a vaccine/adjuvant combination that can induce robust immune responses. Malaria is a debiltating disease that infects an estimated 550 million people annually on a worldwide basis. One protein based vaccine candidate that holds promise in preventing malaria is Merozoite Surface Protein 1 (MSP1). MSP1 is a surface protein found on merozoites of the erythrocytic stage of Plasmodium falciparum, one of the protozoans that cause malaria. Recombinant MSP1, in the form of smaller fragments called MSP1-42 or MSP1-19, is a highly effective human blood stage malaria vaccine. Vaccinations with MSP1-42 in animal models have demonstrated protection but required the use of a potent adjuvant such as the oil-based Complete Freund's Adjuvant (CFA).

Despite demonstration of protective immunity in animal models, at least one clinical trial using MSP1-42 vaccine showed no significant efficacy in humans. (Ogutu et al., “Blood stage malaria vaccine eliciting high antigen-specific antibody concentrations confers no protection to young children in Western Kenya,” PLoS One 4, 2009:e4708). Other trials have shown similar non-protective results. The apparent failure to elicit protective immunity and/or high levels of parasite inhibitory antibodies in these clinical trials and other approaches may be attributed partially to the adjuvants used, e.g., ASO2A, CPG, and Alum.

Thus, new and alternative strategies need to be explored to expand the portfolio of vaccine delivery platforms. Given that the use of adjuvants in vaccine preparations can result in undesirable side effects ranging from localized inflammation to systemic reactions, adjuvant-free vaccines that produce an effective immune response would be highly desirable.

One potential strategy to accomplish these goals makes use of nanoparticle based delivery systems in an attempt at improving immunogenicity through targeted antigen delivery and/or presentation. Among such particles under evaluation are lipid polymers (eg. PLGA, PGA, PLA) virus-like particles (VLP); Immune Stimulating Complexes (ISCOMS); chitosans; and inorganic particles. Some vaccines, such as a Hepatitis B vaccine and a human papilloma virus vaccine, have been developed utilizing this strategy.

The present invention is a nanoparticle mediated delivery system that produces an effective immune response in a subject. More importantly, the invention achieves the goal of producing an effective immune response without the use of any adjuvants. The present invention is anticipated to be useful for in vitro and in vivo studies as well as for disease therapeutics. In particular, the nanoparticle-mediated delivery system described herein is used for enhanced antibody production, efficient delivery of vaccines and/or drugs, as well as for immunotherapy and gene therapy of diseases such as but not limited to cancer, heart disease, drug addiction, infectious diseases, diseases of the central nervous system, etc.

There are several embodiments of the invention. One embodiment is a vaccine for vaccinating an animal (e.g., mammals—including humans, avians) against a pathogen. The vaccine comprises a nanostructure composition which comprises a nanospecies, a polymer encapsulating the nanospecies, and an immunogen attached to the polymer encapsulated nanospecies. The immunogen is chosen such that it is capable of initiating an immunological response in the animal when used in the practice of the invention. The vaccine is capable of producing the immunological response in the absence of an adjuvant.

Another embodiment of the invention is a vaccine for vaccinating an animal (e.g., mammals—including humans, avians) against a pathogen. The vaccine comprises a nanostructure composition which comprises a nanospecies, a polymer encapsulating the nanospecies, and an immunogen. The immunogen is chosen such that it is capable of initiating an immunological response in the animal when used in the practice of the invention. The vaccine is capable of producing the immunological response in the absence of an adjuvant.

Another embodiment of the invention is a method of vaccinating an animal. The method comprises providing a nanostructure comprising a nanospecies, a polymer encapsulating the nanospecies, and an immunogen attached to the polymer. The method further comprises administering to the animal a quantity of the nanostructure sufficient to initiate an immunological response against the immunogen.

A still further embodiment of the invention is a method for eliciting an enhanced immunological response in an animal. The method comprises administering a nanostructure to an animal. The nanostructure comprises a nanospecies, a polymer encapsulating the nanospecies, and an immunogen capable of stimulating an immunological response in an animal.

### DESCRIPTION OF FIGURES

The present embodiments are illustrated by way of example and not limitations in the figures of the accompanying drawings, in which:

FIG. 1 illustrates an exemplar embodiment of a nanostructure that can be used in the practice of the invention.

FIG. 2A-C illustrates antibody titers produced in accordance with the invention using quantum dot (QD) based nanoparticles.

FIG. 3 illustrates the uptake of QD based nanostructures by dendritic cells.

FIG. 4 is a picture of a gel electrophoresis of rMSP1-QD nanostructures.

FIG. 5 is a graph showing antigenicity ofrMSP1-QD nanostructures (open circles) and unconjugated nanoparticles (filled circles) against MSP1-42 specific monoclonal antibody.

FIG. 6A-B depicts IL-4 and IFN-γ responses induced by rMSP1-QDs and other adjuvants.

FIG. 7 is a chart illustrating activation of various antigen presenting cells by rMSP1-QDs.

FIG. 8 is graph illustrating cytokine expression by QD stimulated bone marrow dendritic cells (BMDCs).

FIG. 9 includes graphs showing cytokine production by QD stimulated BMDCs.

FIG. 10 includes graphs showing chemokine production by QD stimulated BMDCs.

FIG. 11 are pictures of gel electrophoresis of rMSP1 (Panel A) and rMSP1 bound to iron oxide (IO) nanoparticles.

FIG. 12 illustrates antibody titers produced in accordance with the invention using IO based nanoparticles.

FIG. 13 is a photograph of various organs from animal subjects.

FIG. 14 are pictures illustrating nanostructure uptake by antigen presenting cells.

FIG. 15 a chart illustrating activation of various antigen presenting cells by rMSP1-IOs.

FIG. 16 includes graphs showing cytokine production by IO stimulated BMDCs.

FIG. 17 includes graphs showing chemokine production by IO stimulated BMDCs.

FIG. 18 includes graphs showing antigenicity ofrMSP1-IO nanostructures.

FIG. 19 are photographs illustrating attachment of antibodies to cancer cells.

The drawings include copies of color photographs and charts which were submitted with the original application.

### DETAILED DESCRIPTION OF ILLUSTRATIVE EMBODIMENTS

In the following description, for purposes of explanation, numerous details are set forth, such as exemplary concentrations and alternative steps or procedures, to provide an understanding of one or more embodiments of the present invention. However, it is and will be apparent to one skilled in the art that these specific details are not required to practice the present invention.

Furthermore, the following detailed description is of the best presently contemplated mode of carrying out the invention. The description is not intended in a limiting sense, and is made solely for the purpose of illustrating the general principles of the invention. The various features and advantages of the present invention may be more readily understood with reference to the following detailed description taken in conjunction with the accompanying drawings.

As used herein, the term “immunogen” refers to proteins, peptides, nucleic acids, chemicals, virus, bacteria, cells, parts of a pathogen, parts of a virus, parts of a bacteria, parts of a cell, or parts of a tissue from plants and/or animals or their combinations. Proteins can include enzymes, antibodies, antigens, haptens, and the like.

The term “adjuvant” means commercially available compounds that are used in the industry to enhance a biological system's immune response to an antigen. The term includes, but is not limited to, MF59, alum, Montanide ISA51, and ASO2A, among others. Although the term can potentially encompass a number of materials (e.g., anything that stimulates inflammation) those skilled in the art understand the term is used herein in its normal sense and should be interpreted accordingly. The term “adjuvant”, as used herein, is different from and does not include nanospecies, antigens, or polymers used to encapsulate nanospecies.

The term “nucleic acid” is intended to encompass oligonucleotides and all forms and types of DNA and RNA (e.g., siRNA), whether isolated from nature, of viral, bacterial, plant or animal (e.g., mammalian or avian) origin, synthetic, single-stranded, double-stranded, sense, anti-sense, comprising naturally or non-naturally occurring nucleotides, or chemically modified.

The term “nanoparticle” is a general term that encompasses particulate material having a dimension between about 1 nm to about 400 nm, preferably between 1 nm and 300 nm, and most preferably between 2 nm and 200 nm. Particularly preferred nanoparticles have a dimension from 1 nm to 100 nm. The term “nanoparticle” is primarily used to designate the very small size of a material and thus is used as a modifier of components that may be more specifically defined elsewhere. This can lead to circular and overlapping definitions with other terms if the definition of the term “nanoparticle” is taken too literally. For example, a “polymer nanoparticle” is a type of “nanospecies” which is a defined term herein. Those skilled in the art are accustomed to the use of the term “nanoparticle” as a generally descriptive term and the proper interpretation of the term will be clear based upon the context in which it is used.

As used herein, the term “nanoparticle-based delivery system” or “nanoparticle mediated delivery system” refers to nanoparticles chemically or physically complexed with one or more immunogens or other biologically active agents (e.g., drugs, imaging agents, etc.).

The term “nanostructure” generally refers to a nanoparticle having two or more components. As used herein the term “nanostructure” typically describes a structure that comprises a “nanospecies” and one or more other components. For example, a “nanostructure” can be a “nanospecies” that is modified in some manner, such as a “nanospecies” having a polymer coating or an attached component (e.g., an immunogen).

The term “nanospecies” refers to a genus of materials having a dimension between 1 nm and 400 nm, preferably between 1 nm and 300 nm, and most preferably between 1 nm and 200 nm. Particularly preferred nanospecies have a dimension between 1 nm and 100 nm. Preferred nanospecies include, without limitation, inorganic nanoparticles, liposomes, micelles, hydrogels, magnetic nanoparticles, polymer nanoparticles, nanocrystals, quantum dots, nanotubes, carbon based nanoparticles (e.g., so-called “Buckyballs”) and the like. Nanospecies can be, without limitation, spherical, rod-like, tube-like, triangular, square, ring-like, wire-like, star-like, or irregular in shape. Various types of nanospecies that may be utilized in the practice of invention are discussed in more detail below.

The term “complexed” refers to an element, compound, chemical species or substance, or material held with another element, substance, or material in chemical union, as those in the chemical arts will recognize. For example, a nanoparticle can be complexed with a chosen molecule (such as a protein), through charge-charge interactions, covalent or ionic bonds, hydrophobic interaction, hydrogen-bonding, or any combination thereof. As used herein the term complexed also refers to the physical combination of a nanoparticle and a second element (e.g., an immunogen) as in an admixture.

As used herein the term “polypeptide” or “protein” is intended to encompass a protein, a glycoprotein, a polypeptide, a peptide, and the like, whether isolated from nature, of viral, bacterial, plant, or animal (e.g., mammalian or avian) origin, or synthetic, and fragments thereof. A preferred protein or fragment thereof includes, but is not limited to, an antigen, an epitope of an antigen, an antibody, an antigenically reactive fragment of an antibody, and antigens derived from surface proteins of prokaryotic or eukaryotic cells.

The term “biocompatibility compound” means a compound that can be included in a nanostructure to aid the biological function of the nanostructure. Such biocompatibility compounds include, but are not limited to polyethylene glycol (MW about 500 to 50,000 and 1000 to 10,000), dextran, and derivatives such as amino-dextran and carboxy-dextran, and polysaccharides.

The term “pathogen” refers to any biological component (e.g., virus, bacteria, prion, protozoan, cancer cell, etc.) that is capable of creating a disease state in an animal.

As an aid to the reader, the invention will be described in general terms first. Examples illustrating the invention follow the detailed description.

The invention comprises a nanoparticle-based delivery system (hereinafter called “delivery system(s)”) and methods for its synthesis and use. More specifically, the delivery systems described herein can be used to provide an enhanced immunological response in living systems as compared to conventional delivery systems (e.g., vaccine compositions containing adjuvants). In other words, the delivery systems described herein have been shown to provide enhanced immunological response in living systems without the use of adjuvants.

In very general terms, the delivery system according to the invention comprises a biologically active nanostructure. The nanostructure comprises a nanospecies, a polymer structure that preferably encapsulates the nanospecies, and an immunogen capable of stimulating an immunological response in an animal when used in the practice of the invention. In preferred embodiments the nanostructure does not comprise an adjuvant and its administration occurs without the co-administration of an adjuvant. Each of these components, and others, are discussed in greater detail below.

Turning now to the subject of the nanostructure, the nanostructure utilized in the practice of the invention include various nanoparticles that are commercially available from Ocean NanoTech, LLC of Springdale, Ark., which are identified more specifically below and in the Examples. Generally speaking, these types of nanoparticles comprise a nanospecies that is modified to include a polymer coating that enhances the particles' biological function, specifically immunological functions. Similar nanostructures and a method for making them are disclosed in U.S. Pat. No. 7,846,412 to Nie et al. (the '412 patent), which is incorporated by reference in its entirety. The following paragraphs offer a general summary of the '412 patent as an aid to the reader in understanding the general architecture of the overall nanostructure that is utilized in the practice of the invention.

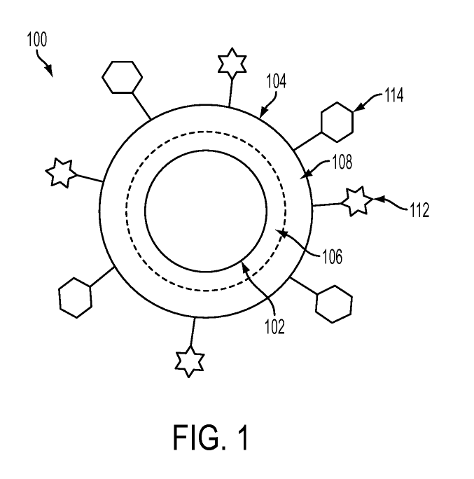

FIG. 1 illustrates an exemplar embodiment of a nanostructure 100 that can be used in the practice of the invention. The nanostructure includes, but is not limited to, a nanospecies 102 having a polymer structure 104 that encapsulates the nanospecies 102. In addition, the nanostructure 100 can include, but is not limited to, an immunogen 114. The nanostructure 100 can include one or more additional components generally represented by element 112. Such additional components include but are not limited to biocompatibility compounds and probes.

The nanostructure can include a number of types of nanospecies such as, but not limited to, semiconductor, metal (e.g., gold, silver, copper, titanium, nickel, platinum, palladium, and alloys thereof), metal oxide nanoparticles (e.g., Cr₂O₃, CO₃O₄, NiO, MnO, CoFe₂O₄, and MnFeO₄, among others), metalloid and metalloid oxide nanoparticles, quantum dots, lanthanide series metal nanoparticles, and combinations thereof. Magnetic nanoparticles (e.g., those having magnetic or paramagnetic properties) can be used as a nanospecies in the practice of the invention. Such particles include, but are not limited to, iron nanoparticles and iron composite nanoparticles (e.g., Fe₂O₃. Fe₃O₄, FePt, FeCo, FeAl, FeCoAl, CoFe₂O₄, and MnFeO₄). Other exemplary nanospecies include semiconducting nanocrystals, e.g., CdS, CdSe, CdTe, ZnS, ZnSe, CuInS, CuInSe, InP, InAs, In₂Se₃, PbS, PbSe, TbTe, Fe₂O₃, Fe₃O₄.

In general, suitable nanospecies for use in the practice of the invention can also include nanospecies with: a) a single atomic species, e.g., carbon (e.g., carbon nanotubes), Sc, Ti, V, Cr, Mn, Fe, Co, Ni, Cu, Zn, Ga, Ge, Y, Zr, Nb, Mo, Tc, Ru, Rh, Pd, Ag, Cd, In, Sn, Sb, W, Re, Qs, Ir, Pt, Au, Pb, Bi, and Ta; b) two atomic species, e.g., CaF₂, BaF₂, MgO, MgS, BBr₃, B₂O₃, BN, B₄C, Al₂O₃, AlN, SiO₂, SiC, SiN, Si₃N₄, TiO₂, TiC, TiN, V₂O₅, CrO₃, MnS, MnO₂, MnO, Mn₂O₃, Fe₂O₃, Fe₃O₄, FeS, CoO, Co₂O₃, Co₃O₄, NiO, Ni₂O₃, Cu₂O, CuO, CuS, ZnS, ZnO, GaAs, GaP, GaN, GeO₂, GeTe, GeSe, As₂O₃, SeO₂, Y₂O₃, ZrO₂, ZrC, Nb₂O₅, MoO₃, TcO₂, Ru₂O₃, RhO, PdS, AgCl, AgBr, AgI, Ag₂S, Ag₂O, CdS, CdSe, CdTe, CdO, InP, InAs, In₂O₃, In₂S₃, SnO₂, SnS₂, Sb₂O₃, TeO₂, Ta₂O₅, LaB₆, La₂O₃, HfO₂, W₂O₃, WS₂, ReO₂, OsO₂, OsO, HgS, HgO, TIO₃, TIP, PbO, PbO₂, PbS, PbSe, PbTe, Bi₂O₅, Gd₂O₃, UO₂, Eu₂O₃, CeO₂, Nd₂O₃, Pr₂O₃, Pm₂O₃, Sm₂O₃Tb₂O₃, Dy₂O₃, Ho₂O₃, Er₂O₃, Tm₂O₃, Yb₂O₃, Lu₂O₃, YF3, YbF₃, ErF₃, GdF₃, UF₄, EuF₃, NdF₃, PrF₃, PmF₃, SmF₃, TbF₃, DyF₃, Ho₂O₃, TmF₃, LuF₃, and LaF₃; c) three atomic species, e.g., AlOOH, Al(OH)₃, BaTiO₃, SrTiO₃, CaCO₃, Ca₃(PO₄)₂, In(OH)₃, LiFePO₄, Mg(OH)₂, MnFe₂O₄, CoFe₂O₄, NiFe₂O₄, InCuS₂, InCuSe₂, CdSeTe, CdZnSe, CdSeS, NaYF₄, BaSO₄, and SrSO₄; and d) four atomic species, e.g., InCuGaS₂, InCuGaSe₂, InCuZnS₂, InCuZnSe₂; and doped NaYF₄. Core/shell structures (discussed in more detail below) are equally applicable using core structures of any of the above nanoparticle compositions and a shell made of Zs and/or ZnSe.

Preferred nanospecies include iron oxide (Fe₂O₃; “IO”) and semiconductor quantum dots such as those described in U.S. Pat. No. 6,468,808 and International Patent Application WO 03/003015, which are incorporated herein by reference.

There are numerous types of quantum dots (QDs) that can be used as a nanospecies in the practice of the invention. Luminescent semiconductor QDs are a particularly preferred QDs for use in applications where visualization of particle location is of benefit. In general, quantum dots include a core and a cap (aka “core/shell” QDs) however, uncapped quantum dots can be used as well. The “core” is a nanometer-sized semiconductor. While any core of the IIA-VIA, IIIA-VA or IVA-IVA, IVA-VIA semiconductors can be used in the context of the present disclosure, the core should be such that, upon combination with a cap, a luminescent quantum dot results. A IIA-VIA semiconductor is a compound that contains at least one element from Group IIB and at least one element from Group VIA of the periodic table, and so on. The core can include two or more elements. In one embodiment, the core is a IIA-VIA, IIIA-VA or IVA-IVA semiconductor that ranges in size from about 1 nm to about 20 nm. In another embodiment, the core is more preferably a IIA-VIA semiconductor and ranges in size from about 2 nm to about 10 nm. For example, the core can be CdS, CdSe, CdTe, ZnSe, ZnS, PbS, PbSe or an alloy.

The “cap” is a semiconductor that differs from the semiconductor of the core and binds to the core, thereby forming a surface layer on the core. The cap can be such that, upon combination with a given semiconductor core a luminescent quantum dot results. The cap should passivate the core by having a higher band gap than the core. In one embodiment, the cap is a IIA-VIA semiconductor of high band gap. For example, the cap can be ZnS or CdS. Combinations of the core and cap can include, but are not limited to the following: (using the convention “core/cap”) CdS/ZnS, CdSe/ZnS, CdSe/CdS, CdTe/ZnS, ZnS/CdS, ZnSe/CdS, CuInS/ZnS, CuInSe/ZnS, PbS/ZnS, and PbSe/ZnS. Other exemplary quantum dots include, but are not limited to, CdS, ZnSe, CdSe, CdTe, CdSeₓTe₁₋ₓ, InAs, InP, PbTe, PbSe, PbS, HgS, HgSe, HgTe, CdHgTe, and GaAs.

The synthesis of quantum dots is well known and is described in U.S. Pat. Nos. 5,906,670; 5,888,885; 5,229,320; 5,482,890; 6,468,808; 6,306,736; 6,225,198, etc., International Patent Application WO 03/003015, (all of which are incorporated herein by reference) and in many research articles. The wavelengths emitted by quantum dots and other physical and chemical characteristics have been described in U.S. Pat. No. 6,468,808 and International Patent Application WO 03/003015 and will not be described in any further detail.

The nanospecies that is chosen for use in the practice of the invention is preferably modified to enhance the biological function of the overall nanostructure. Modifying the nanospecies to impart specific characteristics to the nanospecies and/or the resulting nanostructure is often referred to as “functionalizing” the surface of the nanospecies.

In general, the surface of a nanoparticle can be functionalized or modified to produce a desired physical characteristic such as solubility, biocompatibility, functionality, providing surface moieties for chemical reactions, etc. Exemplary methods for functionalizing or preparing nanoparticle surfaces can be found in: U.S. Pat. No. 7,846,412 to Nie et al.; U.S. Pat. No. 6,649,138, to Adams et al.; U.S. Pat. No. 7,153,703, to Peng et al.; and International Application No. PCT/US2002/015320, to Peng et al.; each of which is incorporated herein in their entirety.

For example, the surface of a nanoparticle can be functionalized by incorporating one or more chemical linkers such as and without limitation: carboxyl groups, amine groups, carboxyl/amine, hydroxyl groups, functionalized polymers, small molecules, and biomolecules. Exemplary functionalization methods are known in the art and can be found in the following references among others: H. Chen, L. Wang, J. Yeh, X. Wu, Z. Cao, Y. A. Wang, M. Zhang, L. Yang, H. Mao. Reducing Non-Specific Binding and Uptake of Nanoparticles and Improving Cell Targeting with an Antifouling PEO-b-PγMPS Copolymer Coating, Biomaterials, 2010, 31(20): 5397-5407; K. Chen, J. Xie, H. Xu, Deepak Behera, M. H. Michalski, S. Biswal, A. Wang, X. Chen. Triblock copolymer coated iron oxide nanoparticle conjugate for tumor integrin targeting. Biomaterials 2009, 30, 6912-6919; Huaipeng Su, Hengyi Xu, Shuai Gao, John David Dixon, Zoraida P. Aguilar, Andrew Y. Wang, Jian Xu, and Jiangkang Wang. Microwave synthesis and applications of nearly monodisperse CdSe-based core/multishell quantum dots for cell imaging. Nanoscale Research Letters. 2010. DOI: 10.1007/s11671-010-9525-1; Zoraida P. Aguilar, Hengyi Xu, John D. Dixon, and Andrew Y. Wang. Blocking Non-specific uptake of engineered nanomaterials. ECS Transactions. 2010. 25 (31), 37-48. DOI: 10.1149/1.3327203 (EI); Hengyi Xu, Zoraida P. Aguilar, Hua Wei, and Andrew Y. Wang. Cell uptake of nanoparticles. ECS Transactions. 2010. 25 (31), 9-17. DOI: 10.1149/1.3327198 (EI); Hengyi Xu, Zoraida P. Aguilar, and Y. Andrew Wang. Quantum dot-based sensors for proteins. ECS Transactions. 2010. 25 (31), 1-8. DOI: 10.1149/1.3327196; and Hengyi Xu, Zoraida P. Aguilar, Huaipeng Su, John Dixon David, Hua Wei, and Andrew Y. Wang. Breast cancer cell imaging using semiconductor quantum dots. ECS Transactions. 2009. 25 (11), 69-77. DOI: 10.1149/1.3236409, each of which is incorporated herein, in their entirety.

In preferred embodiments the nanospecies (and the resulting nanostructures) are water soluble semiconductors, salts, metal oxides, or metal salts. In general, a nanospecies can be made to be water soluble by attaching hydrophilic surface moieties to its surface, through surface modification chemistry known in the art. Such a feature can be desirable to maximize transport of a delivery system into, e.g., blood streams, cells, tissues, and organs. Such functionality can provide enhanced uptake of the delivery system into living tissue compared with traditional adjuvant materials, which are often dissolved in an oil-in-water or water-in-oil emulsions.

In preferred embodiments of the invention, the nanospecies is functionalized by encapsulating the nanospecies with a polymer and attaching biologically active components to the nanospecies via interaction with the polymer coating. Methods for accomplishing such encapsulation and attachment are discussed in the references cited above.

The polymer structure can take several forms depending on the functionality needed. In the practice of the current invention, water solubility is a desired characteristic of the nanospecies and the nanostructure. In addition, choosing a polymer structure that allows the attachment of other components (e.g., immunogens) is also a desired characteristic.

In one embodiment of the invention, the polymer structure is a structure formed of one or two or more polymer components. This embodiment is illustrated in FIG. 1 and discussed at length in U.S. Pat. No. 7,846,412.

Turning now to FIG. 1, in one embodiment, the polymer structure 104 is a structure that comprises a capping ligand 106 and/or a copolymer layer 108.

The capping ligand caps the nanospecies (e.g., quantum dot) and forms a layer on the nanospecies, which subsequently bonds with a copolymer (discussed below) to form the polymer structure. The capping ligand can include compounds such as, but not limited to, an O═PR₃ compound, an O═PHR₂ compound, an O═PHR₁ compound, a H₂NR compound, a HNR₂ compound, a NR₃ compound, a HSR compound, a SR₂ compound, and combinations thereof. “R” can be a C₁ to C₁₈ hydrocarbon, such as but not limited to, linear hydrocarbons, branched hydrocarbons, cyclic hydrocarbons, substituted hydrocarbons (e.g., halogenated), saturated hydrocarbons, unsaturated hydrocarbons, and combinations thereof. Preferably, the hydrocarbon is a saturated linear C₄ to C₁₈ hydrocarbon, a saturated linear C₆ to C₁₈ hydrocarbon, and a saturated linear C₁₋₈ hydrocarbon. A combination of R groups can be attached to P, N, or S. In particular, the chemical can be selected from tri-octylphosphine oxide, stearic acid, and octyldecyl amine. Generally speaking, the capping ligand forms a generally hydrophobic layer adjacent to the nanospecies.

In preferred embodiments, the copolymer layer comprises amphiphilic copolymers, which includes but is not limited to, amphiphilic block copolymers, amphiphilic random copolymers, amphiphilic alternating copolymers, amphiphilic periodic copolymers, and combinations thereof, that are attached to the capping ligand. Examples of each of these types of amphiphilic copolymers are listed in U.S. Pat. No. 7,846,412 starting at column 7, line 41 and continuing to column 15, line 27. Each of the examples listed therein is specifically incorporated herein by reference.

The following illustrative Examples use amphiphilic block copolymers, but other copolymers such as, but not limited to, amphiphilic random copolymers, amphiphilic alternating copolymers, amphiphilic periodic copolymers, and combinations thereof, can be used in combination with block copolymers, as well as individually or in any combination. In addition, the term “amphiphilic block copolymer” will be termed “block copolymer” hereinafter.

The capping ligand and the block copolymer are selected to form an appropriate polymer structure to encapsulate the nanospecies. For example, the block copolymer and the capping ligand and the nanospecies can combine through interactions such as, but not limited to, hydrophobic interactions, hydrophilic interactions, pi-stacking, etc., depending on the surface coating of the nanospecies and the molecular structure of polymers.

In preferred embodiments the amphiphilic copolymer is a block copolymer which includes amphiphilic di- and or triblock copolymers. In addition, the copolymer can include hydrocarbon side chains such as, but not limited to, 1-18-carbon aliphatic side chains, 1-18-carbon alkyl side chains, and combinations thereof. Furthermore, the di or tri block copolymers preferably have at least one hydrophobic block and at least one hydrophilic block.

In particular, the block copolymer can include an ABC triblock structure having a poly-butylacrylate segment, a poly-ethylacrylate segment, and a poly-methacrylic acid segment, for example. The block copolymer can include a diblock and/or triblock copolymer having two or more different poly-aliphatic-acrylate segments. In addition, the block copolymer can include a diblock and/or triblock copolymer having two or more poly-alkyl-acrylate segments.

When completed, the polymer structure formed by the capping ligand and the copolymer provides an encapsulating coating on the nanospecies that has hydrophobic and hydrophilic portions. The interior of the polymer structure is primarily the hydrophobic portion which comprises the capping ligand and the hydrophobic sections of the copolymers. The exterior of the polymer structure is primarily hydrophilic and comprises the hydrophilic ends of the amphiphilic copolymers. This orientation of the polymer structure in embodiments that utilize capping ligand/copolymer encapsulation creates a water soluble nanostructure. Water solubility of the nanostructure is an important aspect of the claimed invention. Additional details regarding the capping ligand and the block copolymer are provided in Example 1 below.

Turning now to the immunogen component of the claimed invention, an immunogen is attached to the nanostructure (i.e., the nanospecies as modified by a polymer coating). The immunogen can be any molecule as previously defined that is capable of being linked to the nanostructure either directly or indirectly via a linker. The immunogen can be attached by any stable physical or chemical association to the nanostructure, directly or indirectly by any suitable means. Functionalized nanoparticles, such as polymer coated nanospecies, can be bound to immunogens by known methods such as ionic interaction, covalent attachment, cross-linking, hydrophobic methods, intercalation, and including methods described in the references above. Chemical linkers can include, without limitation, surface-bound moieties having carboxyl groups, amine groups, carboxyl/amine, functionalized polymers, small molecules, or biomolecules available for bonding to a chosen drug/vaccine. Processes for functionalizing nanoparticles are disclosed in the references provided herein.

In preferred embodiments the immunogen is attached to the nanostructure via attachment to the polymer encapsulating the nanospecies. The immunogen can be primarily disposed on the surface of the functionalized nanoparticle (i.e., the polymer encapsulated nanospecies) as discussed in U.S. Pat. No. 7,846,412 or it can be incorporated into the matrix of the polymer that encapsulates the nanospecies. In embodiments that utilize a capping ligand and a copolymer to form the encapsulating polymer structure, the immunogen can be dissolved in or admixed with the hydrophobic interior of the polymer structure. The latter arrangement may prove beneficial in applications where timed-release of a particular antigen (or a probe or a drug, etc.) is beneficial. In those instances the polymer layer is chosen such that it is compatible with the immunogen (or probe or drug, etc.) and is capable of predictable degradation within a chosen structure of a biological system (e.g., within an antigen presenting cell, within a cancer cell, in the lumen of the blood stream, etc.). Materials suitable as timed-release coatings are known in the art and those skilled in the art capable of choosing the proper coating for a particular application. It is anticipated that in such circumstances the immunogen would be added concurrently with the components of the polymer layer or in a sequence that would provide for deposition of the immunogen within the matrix of the polymer layer.

The scope of the invention also includes an admixture of nanoparticles/nanospecies and an immunogen capable of producing a desired biological or immunological result. In another embodiment, the immunogen can be mixed with or combined physically with the nanoparticles/nanospecies, existing instead as dissolved species in an aqueous admixture.

Protocols for conjugating immunogens (and probes and target molecules) to nanoparticles/nanospecies are known to those skilled in the art and are discussed in several references, including but not limited to: Pusic, et al., “Blood Stage Merozoite Surface Protein Conjugated to Nanoparticles Induce Potent Parasite Inhibitory Antibodies”, Vaccine, 2011, 29(48): 8890-8908; Xu, et al., “Antibody conjugated magnetic iron oxide nanoparticles for cancer cell separation in fresh whole blood”, Biomaterials, 2011, 32(36):9758-9765. The Xu reference discusses bioconjugation with anti-HER2 antibodies, which are related to a human cancer, and are discussed in the Examples below. The Examples also set forth specific conjugation protocols.

As mentioned previously, alternative embodiments of the nanostructure used in the practice of the invention can include biocompatibility components and probes. In embodiments that utilize a probe, the probe molecule is attached to the surface of the nanostructure in a manner similar to the attachment of the immunogen. Typically, a probe has an affinity for one or more target molecules (e.g., cancer cell) for which detection (e.g., determining the presence of and/or proximal position within the vessel (body)) is desired.

The probe molecule and the target molecule can include, but are not limited to, polypeptides (e.g., proteins such as, but not limited to an antibody (monoclonal or polyclonal)), nucleic acids (both monomeric and oligomeric), steroids, purines, pyrimidines, drugs (e.g., small compound drugs), ligands, or combinations thereof. The nanostructure can include two or more probes used to treat a condition and/or disease.

The present disclosure provides methods of fabricating the nanostructures. See, Current Opinion in Biotechnology 2002, 13, 40-46; Nature Biotechnology 2004, 22, 969-976 both of which are incorporated herein by reference. An exemplary method is described in Examples 1 and 2 below.

The nanostructures discussed herein can be included in a porous material such as, but not limited to, a mesoporous material (e.g., a pore diameter of about 1 to 100 nanometers (nm)), a macroporous material (e.g., a pore diameter of greater than about 100 nm), and a hybrid mesoporous/macroporous material. The porous material can be made of a material such as, but not limited to, a polymer, a copolymer, a metal, a silica material, cellulose, ceramic, zeolite, and combinations thereof. The preferred porous materials are silica materials and polystyrene and polystyrene co-polymers (e.g., divinylbenzene, methacrylic acid, maleic acid). The shape of the porous material can be, but is not limited to, spherical, cubic, monolith (i.e., bulk material), and two dimensional and three dimensional arrays. The preferred shape of the porous material is spherical (e.g., silica beads and polymer beads (e.g., chromatographic beads), ceramic, and molecular sieves).

Although the nanostructure utilized in the practice of the invention has been discussed in some detail above, one need not fabricate nanospecies in order to practice the invention. Nanospecies suitable for use in the practice of the invention are commercially available from Ocean NanoTech, LLC, of Springdale, Ark. www.oceannanotech.com. In particular, suitable nanospecies include, but are not limited to, the following products from the Ocean NanoTech, LLC catalog: (note: IOs is an abbreviation for iron oxide nanoparticles) (1) Affinity IOs with Antibodies, Protein G or Streptavidin; (2) Passive IOs with PEG or Positive Charge Coatings; (3) Active IOs with carboxylic acid, amine, or NTA-Ni; (4) Passive QDs with PEG or Positive Charge Coatings; (6) Active QDs with Carboxylic Acid, Amine, or NTA-Ni; and lyophilized nanoparticles (e.g., freeze-dried nanoparticles).

The following Examples illustrate the bio-effectiveness of the claimed invention. In particular, the Examples provide data in support of the use of the invention as a vaccine for vaccinating an animal (including humans) against a pathogen in which the vaccine comprises a nanostructure composition comprising a nanospecies; a polymer encapsulating the nanospecies; and an immunogen. The Examples also provide data in support of the use of the invention as a method of eliciting an immunological response in an animal and a method of vaccinating an animal (including humans). More specifically, the Examples demonstrate that administering a nanostructure to an animal wherein the nanostructure comprises a nanospecies, a polymer structure encapsulating the nanospecies, and an immunogen capable of stimulating an immunological response in the practice of the invention, will elicit a desired immunological response in the animal (e.g., the production of immunoglobulins and a T-cell response). Furthermore, this immunological response occurs in the absence of the administration of any adjuvant either as part of the nanostructure or separately. The Examples will demonstrate that it is capable of eliciting an immune response in primates and is thus a likely candidate for use in humans.

The Examples will illustrate that the claimed invention produces an immunological response that incorporates multiple segments of the immune system and thus is suitable for use as a method of vaccinating an animal by providing a nanostructure wherein the nanostructure comprises a nanospecies; a polymer encapsulating said nanospecies; and an immunogen; and administrating to the animal a quantity of the nanostructure sufficient to initiate an immunological response against the immunogen. In particular, the method of vaccinating is potentially useful in prophylactic vaccinations and post-exposure vaccinations. More specifically, the Examples illustrate that the methods according to claimed invention results in the activation of cellular components of the immune system (e.g., macrophages, T-cells) and the production of biologically active and effective immunoglobulins and the production/release of various cytokines and chemokines targeted to a specific antigen. This ability to activate the immune system to attack a specific antigen indicates that the claimed invention is particularly well suited for immunotherapy applications, specifically cancer immunotherapy where the immunogen used is a cancer specific antigen or other compound, protein, or chemical that is a suitable target of cancer treatment.

The following examples illustrate certain advantages and features but in no way limits the scope of the concepts disclosed herein. Typical scientific methods, procedures, and techniques are described, however, it should be understood that alternatives may also be used.

### Example 1

The results of Example 1 are also discussed in Pusic, et al., Blood stage meroziote surface protein conjugated to nanoparticles induce potent parasite inhibitory antibodies, Vaccine 29 (2011) 8898-8908, which is incorporated by reference in its entirety. Water soluble nanoparticles were tested as a vaccine vehicle/platform to enhance the immunogenicity of antigens in adjuvant-free immunizations using malaria parasite recombinant blood stage merozoite protein, rMSP1-42 as a model vaccine candidate. The term “adjuvant-free immunization” as used herein refers to immunizations free from conventional adjuvants such as Freund's Complete Adjuvant, which are usually mixed in the presence of oil. Specifically, a delivery system including nanoparticles less than 10 nanometers (nm) bound to recombinant malaria vaccine antigen, rMSP1-42, was tested as a malaria vaccine delivery platform.

In this exemplary embodiment, water soluble CdSe/ZnS core/shell nanospecies were surface modified with carboxyl groups and bound to an antigen to form a nanostructure. The QDs utilized in this Example were CdSe/ZnS QDs commercially available from Ocean NanoTech, LLC under catalog identifier QSH. These QDs are functionalized with a polymer coating incorporating a hydrophobic protection structure such as those described previously. It will be understood that nanostructures of different composition are equally contemplated, e.g., Fe₂O₃, Au, Cu, etc., and the choice of which type of nanostructure to use as a delivery platform may be based on a combination of factors such as immunogenicity and safety profiles.

An rMSP1-quantum dot complex (hereinafter rMSP1-QD) induced higher antibody titers compared with the conventional Freund's complete adjuvant (FCA) and Montanide ISA51. The mean titer induced by the rMSP1-QD complex was over two orders of magnitude greater than those observed using CFA and ISA51 adjuvants. Moreover, the antibody levels elicited in mice were higher than any other adjuvants previously tested with MSP1 vaccines. (See Hui et al., “Biological activities of anti-merozoite surface protein-1 antibodies induces by adjuvant-assisted immunizations in mice with different immune gene knockouts,” Clin. Vaccine Immunol. 15, 2008: 1145-1150; and Hui et al., “Adjuvant formulations possess differing efficacy in the potentiation of antibody and cell mediated responses to a human malaria vaccine under selective immune genes knockout environment,” Int. Immunopharmacol. 8, 2008: 1012-1022.) Results from antibody sub-class determination and ELISPOTs showed that rMSP1-QD immunizations potentiated a balanced TH1/TH2 response. Without wishing to be bound by theory, while the importance of TH1 versus TH2 response in anti-MSP1 mediated immunity has yet to be established, the balance between TH1 and TH2 responses may be important against other infectious diseases. (See, e.g., Infante-Duarte and Kamradt, “Th1/Th2 balance in infection,” Springer Semin. Immunopathol. 21, 1999: 317-338; and Quinnell et al., “The immunoepidemiology of human hookworm infection,” Parasite Immunol. 26, 2004: 443-454.)

Equally significant was the ability of rMSP1-QDs to elicit 100% response in outbred mice, independent of immunization route. It is believed that this level of generalized responsiveness could only have been achieved previously with a very potent adjuvant such as CFA.

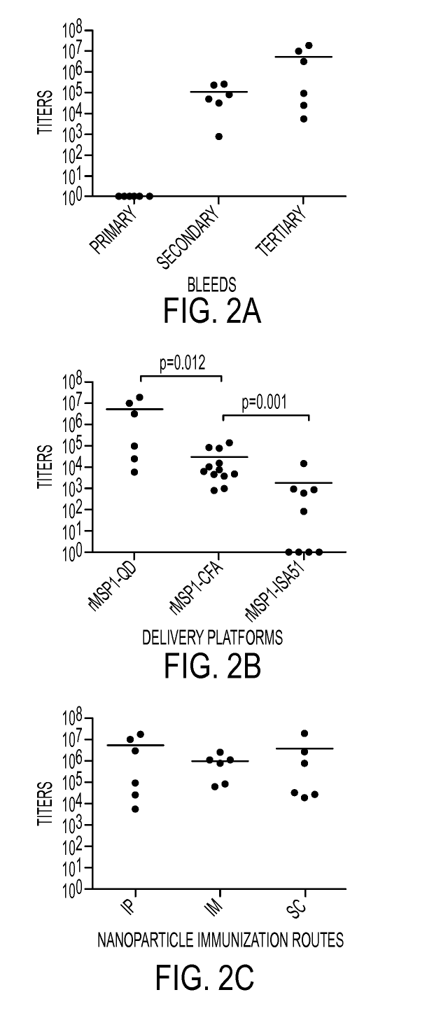

Referring now to FIG. 2, ELISA antibody response against MSP1-19 in SW mice immunized with recombinant MSP1 is shown. Panel A in FIG. 2 shows antibody titers of mice vaccinated (IP) with rMSP1-QD (results of primary, secondary, and tertiary bleeds shown). Panel B in FIG. 2 shows antibody titers of mice vaccinated with different adjuvant/delivery platforms (rMSP1-QD, rMSP1-CFA, and rMSP-1-ISA51) (results of tertiary bleeds are shown). Panel C in FIG. 2 shows antibody response in mice vaccinated with rMSP1-QDs via different routes (intra-peritoneal (i.p.), intra-muscular (i.m.), and sub-cutaneous (s.c.)) (results of tertiary bleeds are shown). In FIG. 2, horizontal bars indicate mean antibody titers; significant differences in ELISA titers among vaccination groups are indicated with p-values (Mann-Whitney test). The data shown in FIG. 2 indicate that the lower toxicity adjuvant, ISA51, induced only 50% of the response induced by the more potent rMSP1-QD complex. Of note is the requirement of two immunizations to induce the high level of response observed with rMSP1-QDs in the non-optimized study. Further optimization of the concentrations of the QD platform, particle size, and surface coating may lead to induction of similar levels of immunogenicity with a single immunization.

Studies have shown that the levels of parasite inhibitory anti-MSP1 antibodies correlate with immunity. In this context, the antibodies produced against rMSP1-QD exhibited greater potency than those produced against rMSP1-CFA and rMSP1-ISA51. Antibodies from rMSP1-QD immunized mouse sera were highly inhibitory against parasite growth (81%), whereas antibodies induced by CFA and ISA51 were completely ineffective.

In some studies, the route of immunization has been shown to play a role in the outcome of immune responses. Referring to FIG. 2C, the rMSP1-QD biomolecule delivery system elicited similar high antibody titers and parasite inhibitory antibodies whether delivered via i.p., i.m., or s.c. routes. Thus, the potency of the rMSP1-QD delivery platform is substantially independent of immunization route. It can be reasonably expected that non-parenteral routes, i.e. intra-nasal and oral administrations are equally or nearly equally effective.

Parallel toxicity evaluations were performed on the immunized mice by examining the plasma levels of Glu, BUN, Na, Cl, TCO2, AnGap, Hct, Hb, pH, PCO2, HCO3, BEecf, and by histological studies of kidney sections. Results showed no significant deviations of these laboratory values and histological findings from non-immunized mice (data not shown).

In general, one advantage of QDs as a delivery platform is the ability to induce antibody and T cell responses without the addition of any adjuvants. However, it is possible that incorporation of adjuvants such as CpG and other TLR ligands to the nanoparticle delivery system could further increase its potency, which may allow for dose sparing administration of the complexed vaccines. In general, another advantage of nanoparticles as a delivery platform is the ability to incorporate large polypeptide antigens, e.g., the MSP1-42.

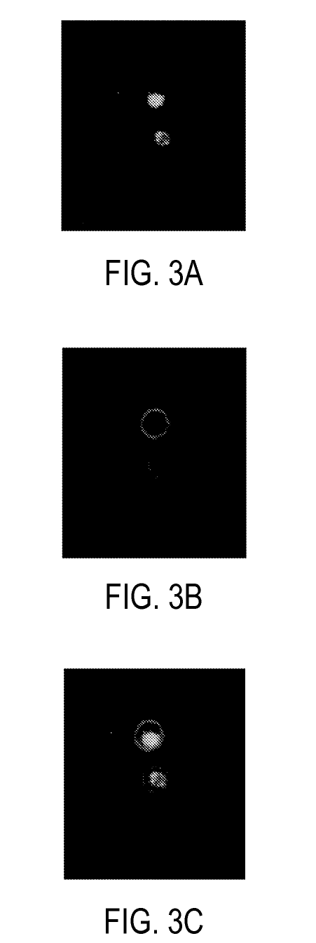

Using mean diameter sizes less than 15 nm, nanoparticle suspensions of the type described herein behave as ‘true’ solutions and thus may readily disperse and penetrate tissues to reach key immunological sites. FIG. 3 shows particle uptake studies with bone marrow derived dendritic cells and indicates that nanoparticles with mean diameters less than 15 nm can be highly effective when they are readily taken up by antigen presenting cells (APCs).

It will be understood that various modifications and optimizations to the procedures and parameters disclosed herein can be made to further increase the immunogenicity of this platform. For example, the method of binding nanoparticles to biomolecules, orientation of the antigen (e.g., either N-terminal or C-terminal binding), and/or differences in animal species response may be modified to optimize immunogenicity. The nature of the nanoparticles, e.g., their type, size, composition, and surface modifications can be modified to optimize effect on the vaccine or drug immunogenicity.

### Experimental Parameters and Procedures

### Mouse Strain

Outbred Swiss Webster (SW) mice (female, 6-8 weeks old) were obtained from Charles River Laboratory (Wilmington, Mass.). The use of mice was approved by the University of Hawaii's Institutional Animal Care and Use Committee.

### Recombinant MSP1-42 (rMSP1)

A truncated version of MSP1-42 was expressed in Drosophila cells and purified by affinity chromatography generally following the procedure disclosed in Chang et al., “A carboxy-terminal fragment of Plasmodium falciparum gp195 expressed by a recombinant baculovirus induces antibodies that completely inhibit parasite growth,” Journal of Immunology 149, 1992: 548-555. This recombinant MSP1-42 has been shown previously to induce parasite inhibitory antibodies.

### Synthesis of Nanoparticle-rMSP1-42 Delivery System

The rMSP1-QD delivery systems were prepared using N-hydroxysulfosuccinimide sodium salt (sulfo-NHS) and 1-ethyl-3-(3-dimethylaminopropyl) carbodiimide (EDC) covalent coupling chemistry. Water soluble QDs with carboxyl groups on the surface (4 μM aqueous solution) were activated by incubating with sulfo-NHS (molar ratio 2000:1) and EDC (molar ratio 2000:1) for 5 minutes in borate buffer, pH 7.4, after which 2 mg of rMSP1-42 was added, vortexed thoroughly, and reacted for 2 hours at room temperature. At the end of 2 hours, the reaction was quenched by adding 5 μL of a quenching buffer, an aqueous borate buffered solution at pH 9.5+/−0.1 (Catalog #QB, Ocean Nanotech, LLC, Springdale, Ark.) and mixed for an additional ten minutes. The rMSP1-QD complexes were stored at 4° C. for about 12 hours and purified by ultra centrifugation using a Beckman ultracentrifuge machine (Beckman, USA).

The water soluble rMSP1-QD complex and unbound (i.e., free) QDs were evaluated by agarose (1.5%) gel electrophoresis in Tris-acetate-EDTA (TAE) buffer at pH 8.5. For each well, 20 μL of 100 nM QD aqueous samples were mixed with 5 μL of 5×TAE loading buffer (5×TAE, 25% (v/v) glycerol and 0.25% (w/v) Orange-G at pH 8.5). The gel was resolved at 100 V for 30 min (PowerPak Basic, Bio-Rad, USA) and then imaged with two exposures using a gel imaging system (Alpha Imager HP 2006, Alpha Innotech, USA).

### Immunization of Mice with rMSP1-QD and rMSP1 with Conventional Adjuvants

SW mice (6 per group) were immunized with rMSP1-QDs using the i.p., i.m., and s.c. routes. Injection volume for i.p. and s.c. routes were 100 μL/dose, and 30 μL/dose for the i.m route.

Mice were also immunized via i.p. with rMSP1 emulsified in either CFA/IFA or Montanide ISA51 (the conventional adjuvant). Mice were immunized three times at 21 days intervals. The first immunization included a sub-optimal dose of 2 μg of antigen, followed by two booster injections with an optimal dose of 5 μg of antigen. Sera were obtained through tail bleeds on the 14th day after each immunization.

### MSP1-Specific Antibody Assays

Mouse sera were assayed for anti-MSP1 antibodies (MSP1-19 specific) by direct binding ELISA substantially as described in Chang et al., “Generalized immunological recognition of the major merozoite surface antigen (gp195) of Plasmodium falciparum,” Proc. Natl. Acad. Sci. USA 86, 1989: 6343-6347. The MSP1-19 used for coating ELISA plates were obtained as described in Hui et al., “Adjuvant formulations possess differing efficacy in the potentiation of antibody and cell mediated responses to a human malaria vaccine under selective immune genes knockout environment,” Int. Immunopharmacol. 8, 2008: 1012-1022. Plates were coated with MSP1-19 at a concentration of 0.4 μg/mL. Mouse sera were serial diluted in 1% yeast extract, 0.5% BSA in Borate Buffer Saline (BBS). Horseradish peroxidase conjugated anti-mouse antibodies (H & L chain specific) (Kirkgaard and Perry Laboratories, Gaithersburg, Md.) were used as a secondary conjugate at a dilution of 1:2000. Optical density (O.D.) was determined at 405 nm. End point titers were calculated using the serum dilutions that gave an O.D. reading of 0.2, which is greater than 4-fold of background absorbance using pre-immune mouse serum.

### Antigenicity of rMSP1 Conjugated to QD Nanoparticles as Determined by ELISA

Following the same ELISA procedures described in the previous section, serial dilutions of rMSP1-QD and unconjugated QD nanoparticles were made and used for coating ELISA plates. The coated ELISA plates were incubated with mAb5.2 at a concentration of 0.2 ug/uL in 1% yeast extract, 0.5% BSA in BBS, followed by incubation with horse raddish peroxidase conjugated goat anti-mouse antibodies. The O.D. readings for each serial dilution of rMSP1-QD and unconjugated QD were plotted and the levels of reactivity were compared to the standard ELISA reactivity of mAB 5.2 against unconjugated rMSP1.

### Isotype-Specific ELISAs

The immunoglobulin isotypes of the anti-MSP1-19 specific antibodies were determined by isotype specific ELISAs as described in Hui et al., “Biological activities of anti-merozoite surface protein-1 antibodies induced by adjuvant-assisted immunizations in mice with different immune gene knockouts,” Clin. Vaccine Immunol. 15, 2008: 1145-1150. Goat anti-mouse-IgG1 and IgG2a (Southern Biotechnology, Birmingham, Ala.) were used at a dilution of 1:4000. Optical density was determined at 405 nm and the OD ratios of IgG1/IgG2a were calculated.

### IFN-γ/IL-4 ELISPOT Assays

ELISPOT assays of splenocytes from immunized mice were generally performed according to methods described in Hui et al., “The requirement of CD80, CD86, and ICAM-1 on the ability of adjuvant formulations to potentiate antibody responses to a Plasmodium falciparum blood-stage vaccine,” Vaccine 25, 2007: 8549-8556. Ninety-six well PVDF plates (Millipore Inc., Bedford, Mass.) were coated with 10 μg/mL of monoclonal antibodies (mAb) against IFN-γ (R4-642) and 5 μg/mL of mAb against IL-4 (11B11) (BD Biosciences, San Diego, Calif.), and incubated overnight at room temperature. Plates were washed with phosphate buffered saline (PBS) and blocked with 10% fetal bovine serum in DMEM for 60 minutes. Mouse spleens were harvested and single cell suspensions of splenocytes were prepared as described in Hui et al., ibid. Purified splenocytes were plated at 0.5×10⁶, 0.25×10⁶, and 0.125×10⁶ cells per well and rMSP1 (4 μg/mL) was added to each well as the stimulating antigen. Positive control wells were incubated with 5 ng/mL of phorbol myristate acetate (PMA) and 1 ng/mL ionomycin. Plates were incubated at 37° C. in 5% CO₂ for 48 hours. Wells were washed and incubated with biotinylated mAb against IFN-γ at 2 μg/mL (XMG1.2), or mAbs against IL-4 at 1 g/mL (BVD6-24G2) (BD, Biosciences, San Diego, Calif.), followed by the addition of peroxidase conjugated streptavidin (Kirkgaard and Perry Laboratories, Gaithersburg, Md.) at a concentration of 1:800. Spots were developed with a solution consisting of 3,3′-diaminobenzidine tetrahydrochloride (DAB) (Sigma-Aldrich St. Louis, Mo., 1 mg/mL) and 30% H₂O₂ (Sigma-Aldrich St. Louis, Mo.) and enumerated microscopically. Data (FIG. 3 of Vaccine Article) were presented as spot-forming-units (SFU) per million of isolated splenocytes.

### In Vitro Parasite Growth Inhibition Assay with Purified Mouse Serum Samples

The ability of mouse sera generated from mice immunized with different rMSP1 formulations to inhibit parasite growth was determined using an in vitro assay. Immunoglobulins from pooled mouse sera samples from each group were then purified as described in Hui, et al., Biological activities of anti-merozoite surface protein-1 antibodies induced by adjuvant-assisted immunizations in mice with different immune gene knockouts. Clin Vaccine Immunol 2008, 15, 1145-50. Antibodies were purified by ammonium sulfate precipitation and followed by dialysis using an Amicon Ultra-10 (Millipore, Billerica, Mass.) with a molecular weight cut off of 100 kDa. Purified antibodies were reconstituted to original serum volume with RPMI 1640. Inhibition assay were performed using sorbitol synchronized parasite cultures (3D7 strain) generally as described in Hui et al., Immunogenicity of the C-terminal 19-kDa fragment of the Plasmodium falciparum merozoite surface protein 1 (MSP1), YMSP1(19) expressed in S. cerevisiae,” J. Immunol 153, 1994: 2544-2553. Synchronized parasite cultures at a starting parasitemia of 0.2% and 0.8% hematocrit were incubated in purified mouse antibodies at an equivalent of 20% serum concentration. Cultures were then incubated for 72 hours with periodic mixing. Parasitemia was determined microscopically by Giemsa staining of thin blood smears and the degree of parasite growth inhibition was determined by comparing parasitemias of immune sera with the corresponding pre-immune sera. (See, e.g., Hui et al., ibid.)

### Dendritic Cell Isolation and QD Uptake Assay

Referring now to FIG. 3, immature bone marrow dendritic cells (BMDC) were isolated from 12-14 week old SW mice. Stromal cells were purified by passage through a cell strainer to remove bone and debris. Red blood cells were lysed using a RBC lysis buffer consisting of 0.15M NH₄Cl, 10 mM KHCO₃, and 0.1 mM EDTA. After washings, BMDCs were plated in 6-well plates (Cell Star, Monroe, N.C.) at a density of 10⁶ cells/mL together with GM-CSF (Peprotech Inc, Rocky Hill, N.J.) at a concentration of 3.33 ng/mL. After 24 hours, cell cultures were further incubated in RPMI 1640 with GM-CSF (6.66 ng/mL) for an additional 48 hours.

Unconjugated QDs (i.e., QDs without rMSP1-42 attached thereto) were introduced at a final concentration of 4 nM to the 3-day old BMDC culture, and incubated for 24 hours at 37° C. Cells were fixed with 1% paraformaldehyde and were labeled with goat anti-CD11c-PE (eBioscience, San Diego, Calif.), at a dilution of 1:2000, for identification and purity assessment. The cells were imaged using a fluorescent microscope (Olympus ix71) with a fluorescent cube containing the following filters: V-N41004 (ex 560 nm and em 585 nm) and V-N41001 (ex 480 nm and em 535 nm).

### Dendritic Cell Activation by QDs

Quantum Dot nanoparticles (4 nM) were introduced to 7-day old BMDCs (53) for 24 hours at 37° C. The cells were harvested and washed twice with FACS buffer (PBS with 2% FBS), fixed with 0.25% PFA for 10 minutes on ice, and stained with cell surface markers: (APC)-labeled anti-CD80, (PE)-labeled anti-MHC II, (AlexaFluor488)-labeled anti-CD11c (eBiosciences, San Diego, Calif.), and (PE-Cy7)-labeled anti-CD86 (Invitrogen, Carlsbad, Calif.). Cells were analyzed using the FACSAria flow cytometer with FACSDiva software (Becton Dickinson, San Jose, Calif.).

### Cytokine Gene Expression by QD Stimulated Dendritic Cells

RNA was extracted from BMDCs (3×10⁶) at 0, 3, 6, and 12 hours after QD or LPS stimulation using the RNeasy Kit (Qiagen, Valencia, Calif.). RNA concentrations were measured and then transcribed in 50 ul reactions using the isc-ript cDNA synthesis kit (Bio-Rad, Hercules, Calif.) following manufacturer's protocol. Real-time PCR reactions using 1 ul of cDNA and iQ SYBR Green Supermix (Bio-Rad, Hercules, Calif.) were run on the MyiQ Single-Color Real Time Detection System (Bio-Rad, Hercules, Calif.). Both forward and reverse primers for TNF-α, TGF-β, IL-12, IL-6, IFN-γ, IL-1β were used at a 10 nM concentration (IDT, Coralville, Iowa). Analysis of gene expression was performed in RT² Profiler PCR.

### Multiplex Assay for Cytokines and Chemokines Detection

The presence of cytokines and chemokines in the supernatants of the BMDCs stimulated with unconjugated QD nanoparticles or with LPS over a 12 hour period were measured using the Milliplex MAP Mouse Cytokine/Chemokine 32 plex assay and Luminex 200 (Millipore Corp, Billerica, Mass.). The following cytokines/chemokines were simultaneously measured: Eotaxin, G-CSF, GM-CSF, IFN-γ, IL-10, IL-12 (p40), IL-12 (p70), IL-13, IL-15, IL-167, IL-1α, IL-1β, IL-2, IL-4, IL-5, IL-6, IL-7, IL-9, <IP-10, KC-like, LIF, LIX, M-CSF, MCP-1, MIG, MIP-1α, MIP-1β, MIP-2, RANTES, TNF-α, VEGF.

### Data Handling and Statistics

Sigma Plot 10 and GraphPadPrizm 4 were used to calculate end point antibody titers. The Mann-Whitney test was used to determine significant differences in antibody titers and isotype ratios among the different test groups.

### Results

### Antigenicity of rMSP1-QD Biomolecule Delivery System

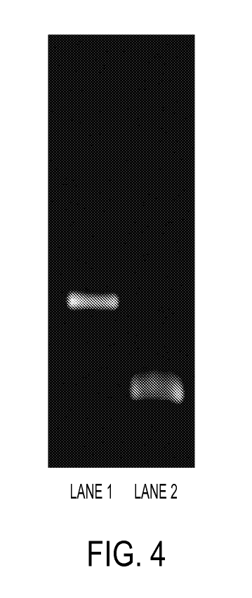

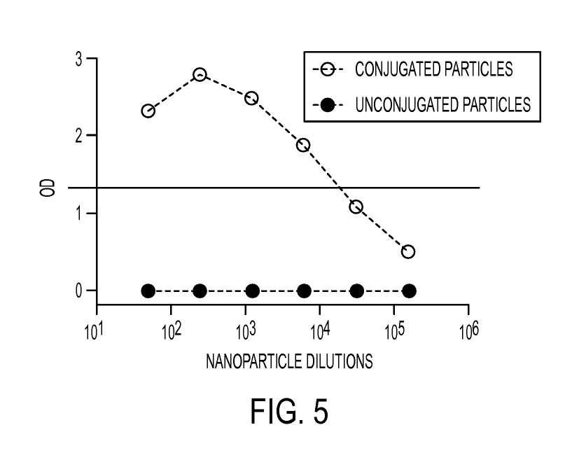

The rMSP1-QD delivery system was tested to determine if the antigen was bound to the nanoparticles, and if the binding processes affected the antigenicity of the rMSP1 biomolecule. Referring now to FIG. 4, bound and unbound QDs were analyzed by 1% agarose gel electrophoresis. rMSP1-QDs (Lane 1) migrated as a single and higher molecular mass band, as compared to the unbound QDs (Lane 2). Without wishing to be bound by theory, this result indicates that the binding process had produced a homogeneous species of rMSP1-QD complexes. The antigenicity of rMSP1 was evaluated by examining the reactivity of the conformation dependent anti-MSP1-42 monoclonal antibody, mAb 5.2, with rMSP1-QD. Referring now to FIG. 5, ELISA titration curves are shown of rMSP1-nanoparticle complex (open circles) and unbound nanoparticles (filled circles) against MSP1-42 specific monoclonal antibody mAb 5.2. The mAb 5.2 strongly recognized the rMSP1-nanoparticle complex, but not the unbound particles. As a reference, an O.D. reading of 1.3 was observed with mAb 5.2 incubated with unbound rMSP1-42 at the plating concentration of 0.4 μg/mL (straight horizontal line in FIG. 5). It is thus highly likely that the antigenicity of the rMSP1 antigen was preserved.

### Immunogenicity of rMSP1-Nanoparticle Complex

The efficacy of QD nanoparticles in enhancing vaccine immunogenicity was compared to conventional adjuvants. Three groups of outbred SW mice were immunized via i.p. with rMSP1-QDs, rMSP1 formulated with CFA, and rMSP1 with ISA51. Immune sera were tested for antibodies against MSP1-19 by ELISA. Vaccine responders were defined as having an ELISA O.D. greater than 0.2 at a 1/50 serum dilution. This was above the O.D. values observed for pre-immune mouse sera. Referring back to FIG. 2A, rMSP1-QDs induced an antibody response in all six mice after two immunizations, resulting in 100% efficacy. In comparison, only five out of ten mice immunized with ISA51 had detectable antibodies, resulting in a 50% response rate. FIG. 2B. All twelve mice that received immunizations with CFA also responded. FIG. 2B.

Comparison of antibody end-point titers of the tertiary bleeds among the three vaccination groups shows that the rMSP1-QDs induced the highest mean antibody titer of 5.3×10⁶ (FIG. 2B) in contrast with the CFA formulation that induced a mean antibody titer of 2.9×10⁴ (p=0.012), and to the ISA51 formulation that induced the lowest mean antibody titer of 1.9×10³ (p=0.001). Thus, immunization of rMSP1-QDs gave antibody titers that were two orders of magnitude higher than the commonly used adjuvants, CFA and ISA51. Despite the high mean antibody titer observed with rMSP1-QD immunizations, there were high and low responders (FIG. 2B) within the group of outbred mice used, as reflected in the broad range of end-point titers.

Still referring to FIG. 2, mice were also immunized with the rMSP1-QD via two other routes, i.m. and s.c. Analysis of the tertiary immune sera revealed that there was 100% response with all three immunization routes. The mean antibody titers induced by s.c. immunizations (3.9×10⁶) were comparable to i.p. immunizations (5.3×10⁶); whereas, i.m. immunizations elicited the lowest mean antibody titer of 0.96×10⁶. (FIG. 2C) However, there were no statistically significant differences in antibody titers among the three routes.

### IgG Isotype Response to MSP1-19

Analyses of the MSP1-19 specific Ig sub-classes (IgG1/IgG2a ratios) in mice immunized with rMSP1-QD (i.p.), rMSP1-CFA (i.p.), and rMSP1-ISA51 (i.p.) showed no significant differences among these groups (Table 1). In addition, comparison of mice immunized via i.p., i.m., and s.c. routes also showed no significant differences. However, rMSP1-ISA51 induced a more polarized IgG1 response as compared to other immunization groups that induced a more balanced IgG1/IgG2a response.

TABLE 1
Immunoglobulin Isotype Specific Antibodies
Against MSP1-19 in Mice Immunized with rMSP1 in
Different Adjuvant/Delivery System^{@}

| Immunogen | IgG1 | IgG2a | IgG1/IgG2a^{†}* |
|---|---|---|---|
| rMSP1-QD (i.p.) | 1.567 ± 0.342 | 0.499 ± 0.132 | 4.147 ± 1.561 |
| rMSP1-QD (i.m.) | 1.431 ± 0.114 | 0.667 ± 0.217 | 3.161 ± 0.882 |
| rMSP1-QD (s.c.) | 1.399 ± 0.132 | 0.579 ± 0.190 | 4.487 ± 1.492 |
| rMSP1-ISA51 (i.p.) | 1.363 ± 0.344 | 0.028 ± 0.009 | 101.8 ± 51.88 |
| rMSP1-CFA (i.p.) | 1.239 ± 0.320 | 0.721 ± 0.314 | 2.989 ± 1.148 |

^{@}Mean O.D. ± SD are shown for IgG1 and IgG2a
^{†}Mean mean ratio of O.Ds IgG1/IgG2a ± SD
*Unpaired t test performed. Significantly different from the rest of the groups

### TH1/TH2

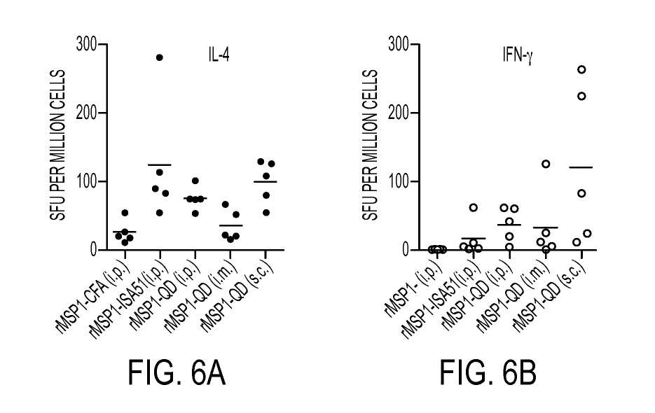

Referring now to FIG. 6, induction of MSP-1 specific IL-4 and IFNγ responses are shown in mice immunized with rMSP1 in five different adjuvant/delivery platforms. ELISPOT analyses of mice immunized with rMSP1-QDs via the i.p., i.m., and s.c. routes showed balanced responses in terms of IL-4 (FIG. 6A) and IFN-γ (FIG. 6B) production. In comparison, rMSP1 formulated with CFA and ISA51 predominantly induced IL-4. There were no significant differences among the groups. Horizontal bars in FIGS. 6A and 6B indicate mean SFU. Mouse splenocytes were harvested 21 days after injection.

### In Vitro Parasite Growth Inhibitory Activity of Recombinant Anti-MSP1-42 Antibodies

Purified mouse antibodies from all immunized groups were tested for their ability to inhibit parasite growth in vitro. As shown in Table 2, the anti-MSP1-42 antibodies obtained from immunizations with rMSP1-QDs via the i.p., i.m., or s.c. route significantly inhibited parasite growth, with inhibition ranging from 73-81%. None of the anti-MSP1-42 antibodies induced by rMSP1-CFA and rMSP1-ISA51 inhibited parasite growth greater than 50%, a level that is considered to be biologically significant.

TABLE 2
In vitro parasite growth inhibition of
purified mouse anti-MSP1 antibodies.

| Pooled Mouse Purified Antibody | |
|---|---|
| (Tertiary Bleeds) | % Parasite growth inhibition* |
| rMSP1-QD (i.p.) | 81% |
| rMSP1-QD (i.m.) | 73% |
| rMSP1-QD (s.c.) | 78% |
| rMSP1-CFA (i.p.) | 17% |
| rMSP1-ISA51 (i.p.) | 0% |

*Mean of two growth inhibition assays.

### Dendritic Cell Uptake of QDs

To better understand the mechanisms by which QDs may enhance immune response, their interaction with dendritic cells in vitro were studied. QDs (emitting at 540 nm) were introduced to 3-day old BMDC cultures and an uptake assay was performed. FIG. 3 shows that BMDCs (CD11c positive) actively internalized the QD nanoparticles. The percent of BMDCs with internalized QDs was approximately 92%.

### Dendritic Cells are Activated by QDs

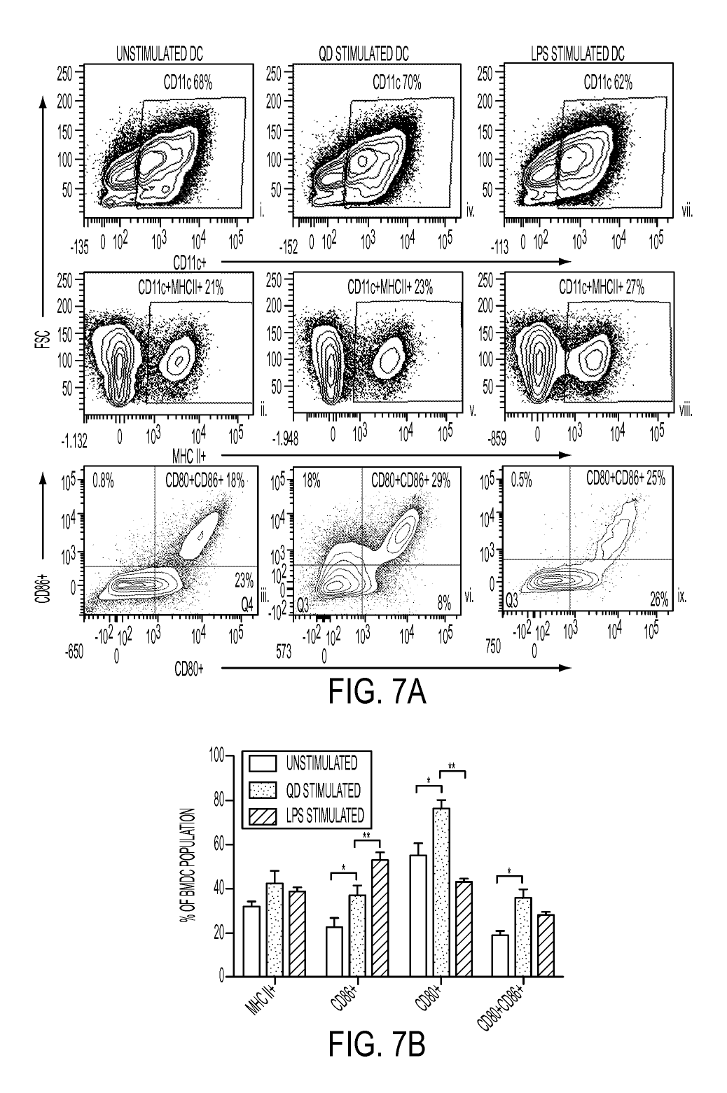

QD nanoparticles were introduced to immature BMDC and the degree of activation was measured by MHC II, CD86, and CD80 expression by flow cytometry. Unstimulated, QD-stimulated, and LPS-stimulated (positive control) dendritic cells were first measured for CD11c and then were further gated for MHC II, CD80, and CD86 activation markers. QD-stimulated, CD11c positive (FIG. 7A, Panel iv) dendritic cells were activated and showed increased expression of MHC II (FIG. 7A, Panel v), CD80, and CD86 (FIG. 7A, Panel vi). QD-stimulated dendritic cells had the highest percentage (42%) of positive MHC II markers compared to unstimulated (32%) and LPS-stimulated (38%) dendritic cells, however these levels were not statistically significant (FIG. 7B). The percentage of single positive CD80 and CD86 cells were statistically higher in QD-stimulated dendritic cells compared to unstimulated dendritic cells with a p value of 0.0172 and 0.0431; respectively (FIG. 7B). Double positive CD80/CD86 expression was also significantly higher as compared to unstimulated dendritic cells (p=0.0086). QD-stimulated dendritic cells induced similar levels of MHC II and double positive CD80/CD86 as LPS-stimulated dendritic cells. However, significantly higher levels of CD80 were observed in QD-stimulated dendritic cells than LPS-stimulated cells (p=0.007), indicating that the QD nanoparticles were able to induce CD80 activation more efficiently than LPS (FIG. 7B). Conversely, LPS stimulated DCs expressed significantly higher CD86 than QD-stimulated DCs, (p=0.0312) (FIG. 7B)

### QDs Uptake Induces Cytokine/Chemokine Production by BMDCs

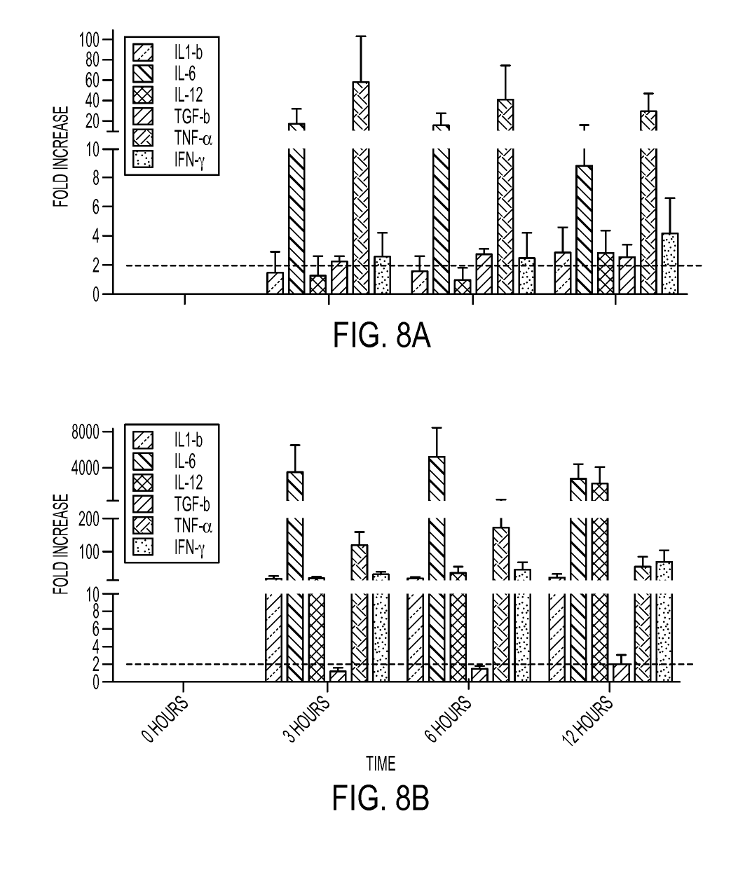

Immature BMDCs exposed to unconjugated QD nanoparticles over a 12-hr period expressed cytokines vital for immune response activation/enhancement. By RT-PCT, QD nanoparticles significantly increased the production of cytokines, TNF-α, IL-6, IFN-γ, IL-12 and TGF-β by more than twofold when compared to levels at 0 hr (FIG. 8, Panel A). QDs uptake primarily led to the increased expression of pro-inflammatory cytokines, TNF-α and IL-6 indicating that immunization with QDs can induce early inflammation similar to LPS stimulation (FIG. 8). On the other hand, LPS-stimulated dendritic cells (DCs) produced a broader array of cytokines assayed, with the sole exception of TGF-β (FIG. 8, Panel B).

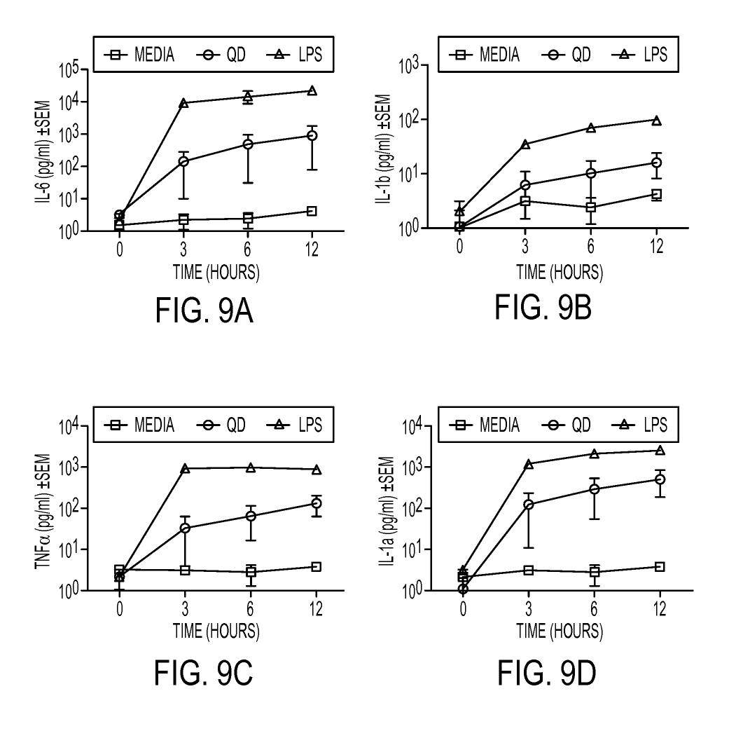

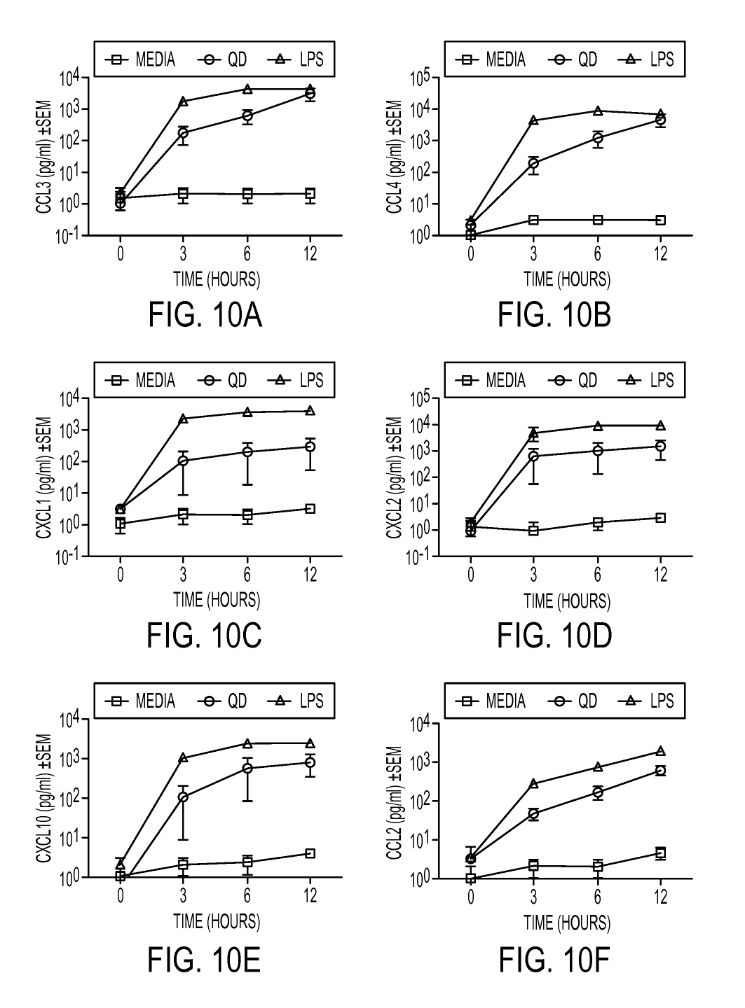

To broaden the assay for cytokine/chemokines a 32-plex Luminex assay was performed. BMDCs stimulated with QD nanoparticles or LPS secreted a number of cytokines (FIG. 9) and chemokines (FIG. 10) over a 12 hour period. In both figures BMDCs (1×10⁶ cells) were incubated with media alone (open squares), QDs (4 μM—open circles), or LPS (100 ng/ml—open triangles) and culture supernatants were collected at 0, 3, 6, and 12 hrs. FIG. 9 shows that QD uptake/stimulation led to higher levels of pro-inflammatory cytokines production; ie. IL-6, TNF-α, IL-1b, and IL-1a in comparison to media alone. A gradual increase of cytokine levels were observed over time with the QD-stimulated BMDC cultures, whereas media alone did not in increase cytokine levels. A number of chemokines were also produced in response to QD stimulation (FIG. 10). Among these, CCL3 and CCL4 were highly expressed and at 12 hours reached the same levels as LPS stimulated BMDCs

A number of illustrative embodiments have been described. Nevertheless, it will be understood that various modifications may be made without departing from the spirit and scope of the various embodiments presented herein. For example, her2 proteins, found in high quantities on the surface of breast cancer cells and other types of cancer, can be attached to nanoparticles to form her2-nanostructures. Similar to the effect of rMSP1-QD, the her2-nanostructure is expected to elicit high titers of antibodies against her2 thereby, sequestering and killing cancer cells that eventually prevent the cancer growth and proliferation. Similarly, attachment of protective antigen (PA) from Bacillus anthracis on nanoparticles to form PA-nanostructure with a targeting receptor towards the lungs when infection is in the lungs will deliver the PA to the lungs to elicit the formation of antibodies against Bacillus anthracis to kill the bacteria and cure the infection.

### Example 2

This example is similar to Example 1 but uses iron oxide (IO; Fe₂O₃) nanoparticles (<15 nm) as a vaccine delivery platform to enhance the immunogenicity of antigens without adjuvants. rMSP1 was used as the model vaccine conjugated to IO nanoparticles to form a rMSP1-IO nanostructure. The IO nanoparticles used in this example are commercially available from Ocean Nanotech, LLC under catalog number SHP. This family of iron oxide nanoparticles are water soluble nanoparticles with diameters ranging from 1 to 100 nm And are carboxyl functionalized on the surface. This example shows that rMSP1-IO was immunogenic in mice and its immunogenicity was equal to that obtained with rMSP1 administered with a clinically acceptable and commercially available adjuvant, Montanide ISA51. Rabbits and Aotus monkeys immunized with rMSP1-IO also achieved comparable immune response that induced significant levels of antibodies with efficient parasite inhibition. There were no apparent local or systemic toxicity associated with IO immunizations. Dendritic cells efficiently took up IO nanoparticles, which led to their activated expression and secretion of co-stimulatory molecules, cytokines and chemokines.

### Experimental Parameters and Procedures

### Mouse, Rabbit, and Non-Human Primates

Outbred Swiss Webster (SW) mice and C57B1/6 mice (female, 6-8 weeks old) were obtained from Charles River Laboratory (Wilmington, Mass.). New Zealand White (NZW) rabbits (female, 8-10 lbs) were obtained from Western Oregon Rabbit Company (Philomath, Or.). Aotus lemurinus trivirgatus karyotype II and III adult monkeys (one female and three males) were colony born and raised at the University of Hawaii's Non-human Primate Facility. Use of all animals was approved by the University of Hawaii's Institutional Animal Care and Use Committee.

### Recombinant MSP1-42 (rMSP1)

The same rMSP1-42 antigen discussed in Example 1 was used.

### Synthesis of Nanostructure-rMSP1-42 Delivery System

The rMSP1-IO conjugates were prepared using N-hydroxysulfosuccinimide sodium salt (sulfo-NHS) and 1-ethyl-3-(3-dimethylaminopropyl) carbodiimide (EDC) covalent coupling chemistry. IOs with carboxyl groups on the surface (5 mg/ml) were activated by incubating with sulfo-NHS (molar ratio 2000:1) and EDC (molar ratio 2000:1) for 5 minutes in borate buffer, pH 7.4, after which 2 mg of rMSP1 was added, vortexed thoroughly, and incubated for 2 hr at room temperature. Following incubation, the reaction was quenched by adding 5 μl of Ocean's quenching buffer, mixed, and incubated for 10 minutes at room temperature. The rMSP1-IO conjugates were then purified/separated by using a SuperMag Separator™ separator (OceanNanoTech, Springdale, Ark.) for 10-24 hours.

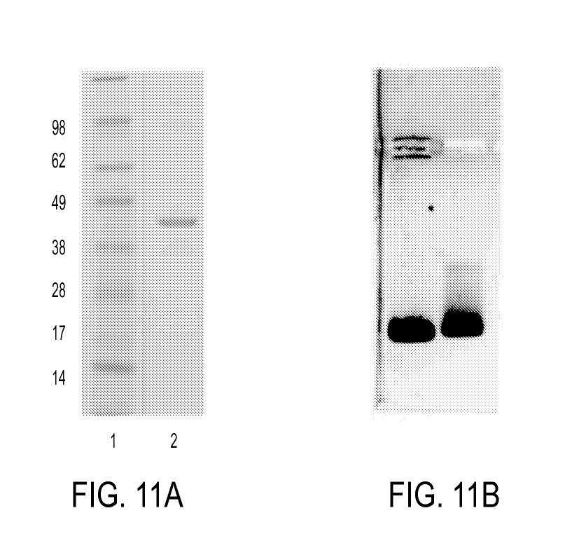

The rMSP1-IO conjugates and unconjugated IOs were evaluated by agarose (1.5%) gel electrophoresis in Tris-acetate-EDTA (TAE) buffer, pH 8.5. For each well, 20 μl of IO samples at 100 nM were mixed with 5 μl of 5×TAE loading buffer 5×TAE, 25% (v/v) glycerol and 0.25% (w/v) orange-G at pH 8.5. The gel was resolved at 100 V for 30 min (PowerPak Basic, Bio-Rad, USA) then imaged with two exposures using a gel imaging system (Alpha Imager HP 2006, Alpha Innotech, USA) (FIG. 11).

### Antigenicity of rMSP1 Conjugated to IO Nanoparticles

Freshly prepared rMSP1-IO and rMSP1-IO stored at 4° C. for 6 and 12 months were used. Serial dilutions of rMSP1-IO were used for coating ELISA plates. MAb 5.2 was used at a 1:200 dilution in 1% yeast extract, 0.5% BSA in BBS. Horseradish peroxidase (HRP) conjugated anti-mouse antibodies (H & L chain specific) (Kirkgaard and Perry Laboratories, Gaithersburg, Md.) at a dilution of 1:2000 were used as a secondary conjugate. Color development was made using the peroxidase substrates, H₂O₂ and 2.2′-azinobis(3-ethylbenzthiazolinesulfonic acid)/ABTS (Kirkgaard and Perry Laboratories, Gaithersburg, Md.). Optical density (O.D.) was determined at 405 nm. ODs for each serial dilution was plotted and the levels of reactivity were compared to the standard reactivity of mAb 5.2 against unconjugated rMSP1.

### Immunizations with rMSP1-IO

Groups of SW mice (n=6) were immunized with rMSP1-IO via intra-peritoneal (i.p), intra-muscular (i.m), and subcutaneous (s.c) routes. Injection volume for i.p and s.c routes were 100 ul/dose (16 ug/dose), and i.m route was 20 ul/dose (5 ug/dose). Mice were also immunized via i.p. with rMSP1 emulsified in either CFA/IFA or Montanide ISA51. Mice were immunized three times at 21 days intervals. The first immunization consisted of a sub-optimal dose of 2 μg antigen, followed by two booster injections with an optimal dose of 5 μg. Sera were obtained through tail bleeds on the 14th day after each immunization.

New Zealand White rabbits were also immunized with rMSP1-IO. Briefly, 0.5 ml/dose (80 ug antigen/dose) of rMSP1-IO was injected intramuscularly into the left and right thighs. A total of four immunizations were given at 4 week intervals. Sera collected 21 days after the last immunization was used in ELISAs and parasite growth inhibition assays. As a control, rabbits were similarly immunized with 50 ug of rMSP1 antigen in 250 ul PBS emulsified with an equal volume of Montanide ISA51 into the left and right thighs.

Aotus lemurinus trivirgatus monkeys (n=4) were immunized with rMSP1-IO, 0.5 ml/dose (80 ug antigen/dose), via the i.m. route. Immunizations were administered three times at 21 day intervals, alternating the right and left thigh. Sera were collected 21 days after the last immunization for ELISAs and parasite growth inhibition assays.

### MSP1-Specific Antibody Assays

Mouse, rabbit, and monkey sera were assayed for anti-MSP1 antibodies (MSP1-42 and MSP1-19 specific) by direct binding ELISA as previously described in Example 1. The MSP1-19 and MSP1-42 used for coating ELISA plates were expressed in yeast as described in Hui, et al., Immunogenicity of the C-terminal 19-kDa fragment of the Plasmodium falciparum merozoite surface protein 1 (MSP1), YMSP1(19) expressed in S. cerevisiae. Journal of Immunology 1994, 153, 2544-2553, and in baculovirus as described in Chang, et al., A carboxyl-terminal fragment of Plasmodium falciparum gp 195 expressed by a recombinant baculovirus induces antibodies that completely inhibit parasite growth. Journal of Immunology 1992, 149, 548-555; respectively. MSP1-19 and MSP1-42 was used to coat the plates at a concentration of 0.4 ug/ml. Sera were serially diluted in 1% yeast extract, 0.5% BSA in Borate Buffer Saline (BBS). HRP-conjugated anti-mouse antibodies (H & L chain specific) (Kirkgaard and Perry Laboratories, Gaithersburg, Md.) were used as a secondary conjugate at a dilution of 1:2000; HRP-conjugated anti-rabbit antibodies (Kirkgaard and Perry Laboratories, Gaithersburg, Md.) were used at a dilution of 1:2000; and HRP-conjugated, anti-Aotus antibodies, provided by Hawaii Biotech Inc, were used at a dilution of 1:16000. Color development was performed by using the peroxidase substrates, H₂O₂ and 2.2′-azinobis(3-ethylbenzthiazolinesulfonic acid)/ABTS (Kirkgaard and Perry Laboratories, Gaithersburg, Md.). Optical density (O.D.) was determined at 405 nm. End point titers were calculated using the serum dilutions that gave an O.D. reading of 0.2, which is greater than 4-fold of background absorbance using pre-immune mouse, rabbit, or monkey serum samples.

### IFN-γ and IL-4 ELISPOT Assays

ELISPOT assays of splenocytes from immunized mice were performed according to methods previously described. Briefly, ninety-six well PVDF plates (Millipore Inc., Bedford, Mass.) were coated with 10 ug/ml of monoclonal antibodies (mAb) against IFN-γ (R4-642) and 5 ug/ml of mAb against IL-4 (11B11) (BD Biosciences, San Diego, Calif.), and incubated overnight at room temperature. Plates were washed with Phosphate Buffered Saline (PBS) and blocked with 10% fetal bovine serum in DMEM for 60 minutes. Mouse spleens were harvested and single cell suspensions of splenocytes were prepared as previously described. Purified splenocytes were plated at 0.5×10⁶, 0.25×10⁶, and 0.125×10⁶ cells per well and rMSP1 (4 ug/ml) was added to each well as the stimulating antigen. Positive control wells were incubated with 5 ng/ml of phorbol myristate acetate (PMA) and 1 ng/ml ionomycin. Plates were incubated at 37° C. in 5% CO₂ for 48 hours. Wells were washed and incubated with biotinylated mAb against IFN-γ at 2 μg/ml (XMG1.2), or mAbs against IL-4 at 1 μg/ml (BVD6-24G2) (BD, Biosciences, San Diego, Calif.), followed by the addition of peroxidase conjugated streptavidin (Kirkgaard and Perry Laboratories, Gaithersburg, Md.) at a concentration of 1:800. Spots were developed with a solution consisting of 3,3′-diaminobenzidine tetrahydrochloride (DAB) (Sigma-Aldrich St. Louis, Mo., 1 mg/ml) and 30% H₂O₂ (Sigma-Aldrich St. Louis, Mo.) and enumerated microscopically. Data were presented as spot-forming-units (SFU) per million of isolated splenocytes.

### In Vitro Parasite Growth Inhibition Assay

The ability of mouse, rabbit, and monkey sera, generated by immunizations with rMSP1-IO, to inhibit parasite growth was determined using the in vitro assay.

For testing mouse serum samples, immunoglobulins from pooled mouse serum samples from each group were purified as previously described. Briefly, antibodies were purified by ammonium sulfate precipitation followed by dialysis using an Amicon Ultra-10 (Millipore, Billerica, Mass.) with a molecular weight cut off of 100 kDa. Purified antibody samples were reconstituted to original serum volume with RPMI 1640 medium and were used at a 20% serum concentration. For testing of rabbit and monkey samples, individual serum samples were heat inactivated, absorbed with normal RBCs, and used at a 30% final serum concentration. Inhibition assays were performed using sorbitol synchronized parasite cultures (3D7 strain) as described. Synchronized parasite cultures at a starting parasitemia of 0.2% and 0.8% hematocrit were incubated in antibody or serum samples for 72 hours with periodic mixing. Culture parasitemias were determined microscopically by Giemsa staining of thin blood smears, and the degree of parasite growth inhibition was determined by comparing the parasitemias of immune sera with the corresponding pre-immune sera as previously described.

### Dendritic Cell and Macrophage Isolation and 10Uptake Assay

Immature bone marrow cells were isolated from 12-14 week old C57B1/6 mice. Inaba et al., Generation of large numbers of dendritic cells from mouse bone marrow cultures supplemented with granulocyte/macrophage colony-stimulating factor. J Exp Med 1992, 176, 1693-1702. Stromal cells were purified by passage through a cell strainer to remove bone and debris. RBC lysis buffer consisting of 0.15 M NH₄Cl, 10 mM KHCO₃, and 0.1 mM EDTA was used in order to remove red blood cells. After washings, bone marrow cells were plated in 6-well plates (Cell Star, Monroe, N.C.) at a density of 10⁶ cells/ml together with either GM-CSF (Peprotech Inc, Rocky Hill, N.J.) at a concentration of 20 ng/ml or with M-CSF (eBioscience, San Diego, Calif.) at a concentration of 10 ng/ml. After 24 hours, cell cultures were incubated in RPMI 1640 with GM-CSF for an additional 8 days for differentiation into dendritic cells (BMDC) or incubated for an additional 6 days in DMEM with M-CSF for differentiation into macrophages. Zhang, et al., The isolation and characterization of murine macrophages. Curr Protoc Immunol 2008, Chapter 14, Unit 14 1. On Day 8, BMDCs in suspension were transferred to new plates and used as the cell source for all subsequent experiments. Szymczak, et al., Antigen-presenting dendritic cells rescue CD4-depleted CCR2−/− mice from lethal Histoplasma capsulatum infection. Infect Immun 78, 2125-37. Experiments were performed using macrophages from Day 6 cultures. Zhang, et al., ibid.

Unconjugated IO nanoparticles were introduced at a concentration of 5 mg/ml to the 8-day old BMDCs or 6-day old macrophages and incubated for 24 hours at 37° C. To first visualize the uptake of iron oxide nanoparticles, BMDCs and macrophages were fixed with 4% paraformaldyhde (PFA) and stained with Prussian Blue (Biopal, Worcester, Mass.) according to manufacture's protocol (http://www.biopal.com/Molday%20ION.htm). The same cells were then stained for surface markers anti-CD11c or anti-CD11b-biotin antibodies (eBioscience, San Diego, Calif.) at a dilution of 1:2000 for one hour, washed, and then further labeled with streptavidin-QDots, which has an emission wavelength of 620 nm (Oceannanotech, Springdale, Ark.), for an additional hour for identification and purity assessment. Cells were then imaged using a fluorescent microscope (Olympus ix71) with a fluorescent cube containing the following filters: V-N41004 (ex560 and em585) and V-N41001 (ex480 and em535).

### Dendritic Cell and Macrophage Activation by IOs

Unconjugated Iron Oxide nanoparticles (5 mg/ml) were introduced to 7-day old BMDCs or 6-day old macrophages for 24 hours at 37° C. Szymczak, et al., Antigen-presenting dendritic cells rescue CD4-depleted CCR2−/− mice from lethal Histoplasma capsulatum infection. Infect Immun 78, 2125-37. The cells were harvested and washed twice with FACS buffer (PBS with 2% FBS) and fixed with 0.25% PFA for 10 minutes on ice. Cells were separated by passing through a magnetic LD column (Miltenyi Biotec Inc., Auburn, Calif.) to obtain an enriched population of cells that have taken up the IO nanoparticles. BMDCs and macrophages were stained with cell surface markers: (APC)-labeled anti-CD80, (PE)-labeled anti-MHC II, (AlexaFluor488)-labeled anti-CD11c or (AlexaFluor488)-labeled anti-CD11b (eBiosciences, San Diego, Calif.), and (PE-Cy7)-labeled anti-CD86 (Invitrogen, Carlsbad, Calif.). Labeled cells were analyzed using the FACSAria flow cytometer with FACSDiva software (Becton Dickinson, San Jose, Calif.).

### Cytokine Gene Expression by IO Stimulated Dendritic Cells and Macrophages

BMDCs and macrophages (3×10⁶ cells) were stimulated with unconjugated IO or LPS (concentration) and RNA was extracted at 0, 3, 6, and 12 hours using the RNeasy Kit (Qiagen, Valencia, Calif.). RNA concentrations were measured and then reversed transcribed in 50 ul reactions using the iScript cDNA synthesis kit (Bio-Rad, Hercules, Calif.) following manufacturer's protocol. Real-time PCR reactions using iQ SYBR Green Supermix (Bio-Rad, Hercules, Calif.) were run on the MyiQ Single-Color Real Time Detection System (Bio-Rad, Hercules, Calif.). Primers for TNF-α, TGF-β, IL-12, IL-6, IFN-γ, IL-1β were used at 10 nM (IDT, Coralville, Iowa). Analysis of gene expression was performed by the ΔΔCt method. Briefly, each sample was normalized to an endogenous control, GAPDH, and fold change for each assayed gene was determined via the ΔΔCt.

### Multiplex Assay for Cytokine Detection

Supernatants from IO and LPS stimulated BMDCs were tested for the presence of cytokines/chemokine over a 12 hour period. Cytokines and chemokines were measured using the Milliplex MAP Mouse Cytokine/Chemokine 32-plex assay (Millipore Corp, Billerica, Mass.) as described. The following cytokines were measured: Eotaxin, G-CSF, GM-CSF, IFN-γ, IL-10, IL-12 (p40), IL-12 (p70), IL-13, IL-15, IL-17, IL-1α, IL-1β, IL-2, IL-2, IL-4, IL-5, IL-6, IL-7, IL-9, IP-10, KC-like, LIF, LIX, M-CSF, MCP-1, MIG, MIP-1α, MIP-1β, MIP-2, RANTES, TNF-α, VEGF.

### Data Handling and Statistics

SigmaPlot 10 and GraphPadPrizm 4 were used to calculate the end point titers. The Mann-Whitney test was used to determine significant differences in antibody responses, and the expression of cell surface activation markers among the test groups. A p value of <0.05 was considered statistically significant.

### Nanoparticles

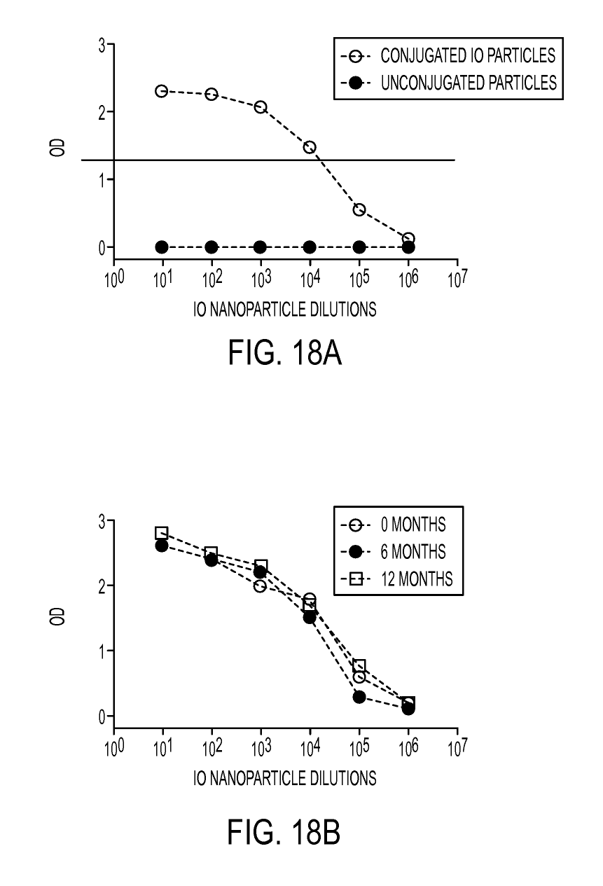

To determine if rMSP1 was successfully conjugated to IO nanoparticles, unconjugated and conjugated IOs were analyzed by agarose gel electrophoresis (FIG. 11A). The rMSP1-IO sample (Lane 2) migrated as a single band and at a higher molecular mass than the unconjugated IO sample (Lane 1), indicating that the conjugation process had successfully produced a homogeneous species of rMSP1-IOs. To evaluate if the chemical conjugation process affected the antigenicity and stability of rMSP1, the reactivity of a conformational dependent anti-MSP1-42 monoclonal antibody, mAb 5.2, with rMSP1-IO was tested. MAb 5.2 strongly reacted with the rMSP1 conjugated to IO nanoparticles but did not recognize the unconjugated IO particles (FIG. 18, Panel A). As a reference, an O.D. reading of 1.3 was observed with mAb 5.2 incubated with unconjugated rMSP1-42 at a plating concentration of 0.4 ug/mL. This suggests that the antigenicity of the rMSP1 antigen was preserved during the conjugation process. The conjugated nanoparticles stored at 4° C. were tested over a period of 12 months for any loss of antigenicity of the rMSP1. The rMSP1-IO was equally reactive with mAb 5.2 at 6 and 12 months post-conjugation (FIG. 18, Panel B), demonstrating the stability of these conjugated IO nanoparticles.

### Immunogenicity of rMSP1-IO in Swiss Webster Mice

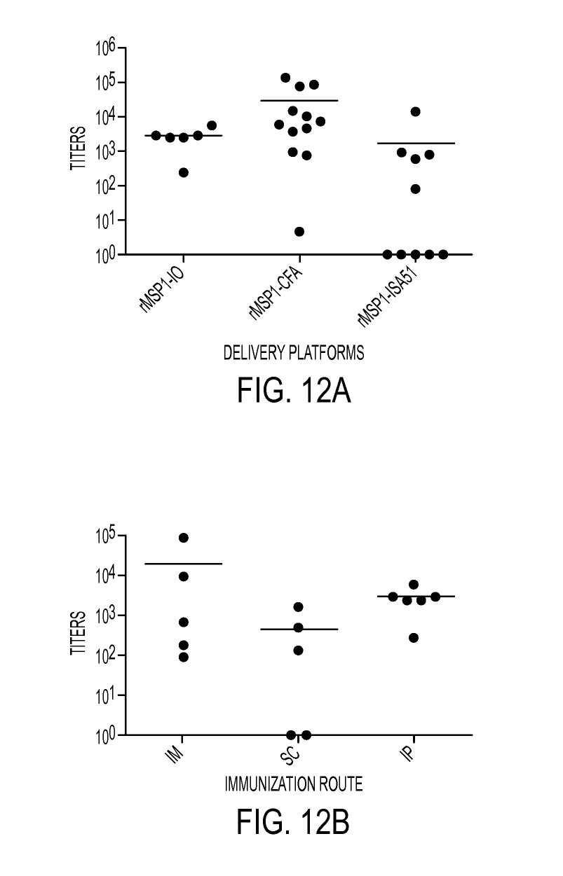

The immunogenicity of rMSP1-IO was compared with conventional adjuvants. SW mice were immunized with rMSP1 conjugated to IO nanoparticles, or formulated with CFA or Montanide ISA51. Immune sera were tested for antibodies against MSP1-19 in an ELISA. Vaccine responders were defined as having an ELISA O.D.>0.2 at a 1/50 serum dilution which was above the O.D. values observed for pre-immune mouse sera. The rMSP1-IO induced an antibody response in all six mice after three immunizations, resulting in a 100% response rate. The same response rate was observed with mice immunized with rMSP1-CFA. However, only five often mice immunized with rMSP1-ISA51 responded, resulting in a 50% response rate (FIG. 12, Panel A). This indicated that IO was more efficient in inducing antibody response that ISA 51 and was as potent as CFA

Comparisons of antibody end-point titers of tertiary bleeds amongst the three vaccination groups showed that rMSP1-IO induced a mean antibody titer of 2.7×10⁻³, whereas the ISA51 formulation induced a lower mean antibody titer of 1.6×10⁻³ (p=0.012). FIG. 12, Panel A. The potent CFA formulation induced the highest mean antibody titer of 2.8×10⁻⁴ that was not significantly higher than rMSP1-IO. Since IO is made of FDA approved chemicals, its ability to induce comparable antibody titer with that of CFA shows potential application in human vaccine delivery. In addition, the ability of IO to induce a uniform antibody titer among the animals tested, unlike CFA and ISA51, makes it a better candidate for vaccine delivery platform.

Mice were also immunized with rMSP1-IO via the i.m. and s.c. routes. Analysis of end-point titers revealed that the mean antibody titers induced by intra-muscular (i.m.) immunization were higher compared to that induced by intra-peritoneal (i.p.) or sub-cutaneous (s.c.) immunizations (FIG. 12), but the difference was not statistically significant. Only immunizations via the i.m. and i.p. routes achieved a 100% response rate. The s.c. immunization resulted in a 60% response rate. (FIG. 12, Panel B).

Sera from rMSP1-IO immunized mice were also tested for their ability to inhibit parasite growth in vitro. Inhibition greater than 50% was considered to be biologically significant. As shown in Table 3, antibodies obtained from rMSP1-IO immunizations via the i.p. and i.m. route significantly inhibited parasite growth at 80% and 74% respectively. In comparison, antibodies from mice immunized with rMSP1 emulsified with CFA and ISA51 were both ineffective in inhibiting parasite growth (Table 3). In addition, IO immunization via the s.c. route was also ineffective at a 37% parasite growth inhibition (Table 3). Based on these results, that IO is an effective vaccine delivery platform because the antibodies produced in its presence inhibits P. falciparum growth whereas those produced with CFA and ISA51 cannot.

TABLE 3
In vitro parasite growth inhibition of
purified mouse anti-MSP1 antibodies.

| Pooled Mouse Purified Antibody | |
|---|---|
| (Tertiary Bleeds) | % Parasite growth inhibition* |
| rMSP1-IO (i.p.) | 80% |
| rMSP1-IO (i.m.) | 74% |
| rMSP1-IO (s.c.) | 37% |
| rMSP1-CFA (i.p.) | 17% |
| rMSP1-ISA51 (i.p.) | 0% |

*Mean of two growth inhibition assays.

### Immunogenicity of rMSP1-IO Nanoparticles in Aotus Monkeys and In Vitro Parasite Growth Inhibition Assay

The ability of monkey sera, generated by immunizations with rMSP1-IO, to inhibit parasite growth was determined using an in vitro assay.

All four Aotus monkeys immunized with rMSP1-IO produced anti-MSP1-42 and anti-MSP1-19 antibodies, with endpoint titers specific for MSP1-42 ranged from 1/2,800 to 1/29,000; and those specific for MSP1-19 ranged from 1/3,000 to 1/24,000 (Table 4). Sera from Aotus monkeys immunized with rMSP1-IO were also evaluated for inhibition of parasite growth as above. All immunized monkeys produced significant levels of parasite growth inhibitory antibodies, ranging from 55% to 100% inhibition (Table 4). This level of inhibition is comparable to studies where Aotus monkeys were vaccinated with MSP1-42-CFA.

TABLE 4
Antibody titers and In vitro Parasite Growth
Inhibition of Monkey Anti-MSP1 Antibodies

| Monkey Serum | Anti-MSP1 Antibody Titers | | % Parasite growth |
|---|---|---|---|
| (Tertiary Bld) | MSP1-42 | MSP1-19 | inhibition |
| Monkey #1 | 2,800 | 3,000 | 82% |
| Monkey #2 | 29,000 | 24,000 | 100% |
| Monkey #3 | 4,500 | 10,000 | 56% |
| Monkey #4 | 10,000 | 20,000 | 66% |

Table 5 is a comparison of the efficacy of the rMSP1-IO mediated antibodies to the QD mediated antibodies referenced in Table 2.

TABLE 5
Immunoactivites of the Antibodies (host animal: SW outbred
mice) against malaria agent P. falciparum

| Injection route | Adjuvant | Parasite Inhibition (%) |
|---|---|---|
| Intraperitoneal, ip | QD | 81% |
| Intraperitoneal, ip | Iron oxide | 80% |
| Intraperitoneal, ip | CFA | 17% |
| Intraperitoneal, ip | ISA51 | 0% |
| Intramuscular, im | QD | 73% |
| Sub-cutaneous, sc | QD | 17% |

### Toxicity Studies Showed No Abnormalities in IO Immunized Animals

Escalating injection doses of IO nanoparticles, up to 4.4 mg per injection, did not cause any abnormalities or changes in the blood chemistries in all four groups of mice tested after each of the three immunizations. Similarly, a more comprehensive test panel of blood chemistry levels in the Aotus monkeys after three rMSP1-IO immunizations revealed no significant deviations from normal ranges. Thus, immunization with IO nanoparticles did not have toxic systemic affects in either animal model.

### Uptake of IO Nanoparticles by Dendritic Cells and Macrophages

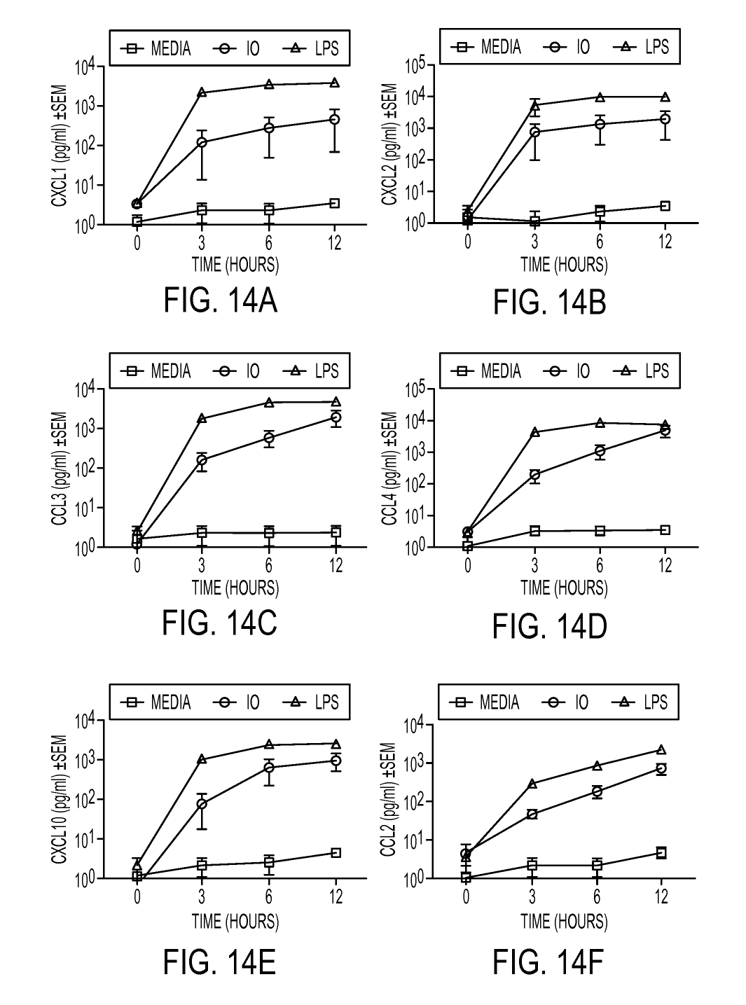

nanoparticles were introduced to 7-day old BMDC cultures and to 6-day old macrophage cultures. BMDCs and macrophages both actively internalized the IO nanoparticles as shown in FIG. 14, Panels A & B. BMDCs were identified by staining for the surface marker, CD11c and the presence of internalized iron oxide particles was identified by Prussian Blue staining. Approximately 89% of the BMDCs internalized IOs. Macrophages were identified by staining for the surface marker, CD11b and approximately 94% of these cells internalized IO nanoparticles as revealed by Prussian Blue staining. Thus, these results indicate that the DCs and Macrophages efficiently uptake the IO and all that is attached to its surface very efficiently

### Dendritic Cell and Macrophage Activation by IOs

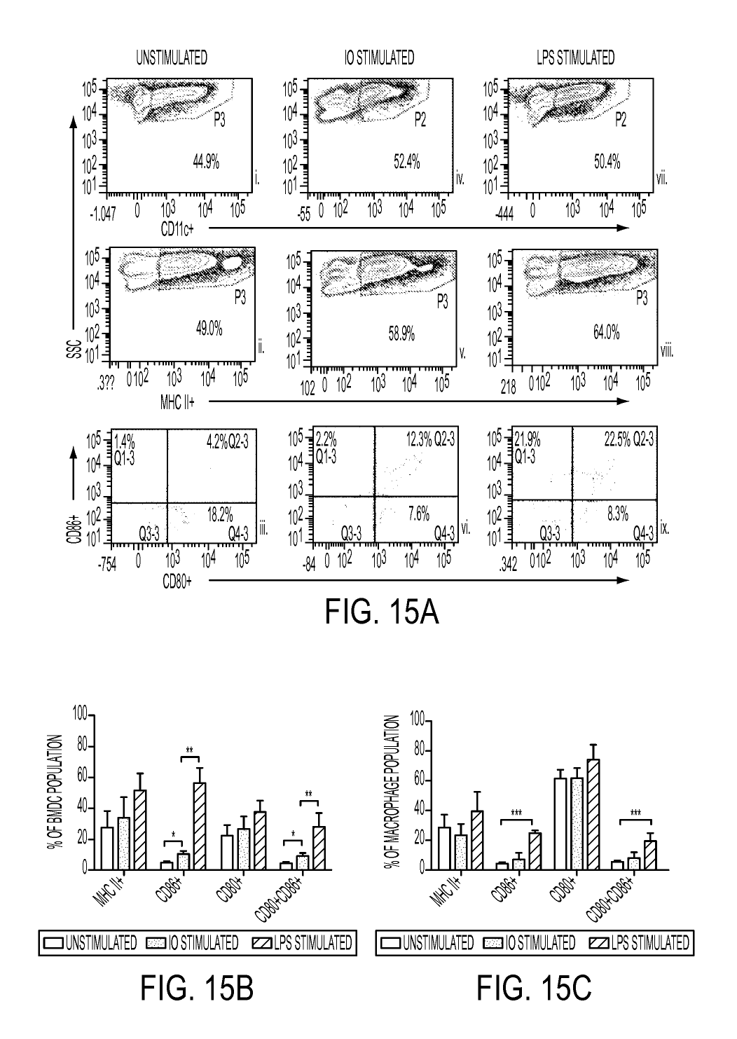

To evaluate the mechanism for the effective immune response, unconjugated IO nanoparticles were introduced to immature BMDCs and macrophages and the degree of activation was determined by cell surface expression of CD86, and CD80 using Flow Cytometry. Unstimulated, IO-stimulated, and LPS-stimulated dendritic cells were first gated for the presence of CD11c, and the CD11c+ cells were analyzed for the expression of activation markers, MHC II, CD86, and CD80. IO-stimulated, CD11c positive dendritic cells (FIG. 15A, Panel iv) were activated and showed an increase in expression of MHC II (FIG. 15, Panel v), CD86, and CD80 (FIG. 12A, Panel vi). IO-stimulated dendritic cells had the highest percentage of MHC II marker (34%) and CD80 marker (28%) as compared to unstimulated dendritic cells (28% and 22% respectively). However, these increases did not reach statistical significance (FIG. 15B). The percentages of CD86+ cells and CD80/86 double positive cells were significantly higher than those observed for unstimulated dendritic cells, with p values of 0.05 and 0.03; respectively (FIG. 15B). LPS-stimulated DCs had significantly higher percentage of CD86+, and CD80/86+ cells than IO-stimulated DCs (p values 0.05 and 0.04 respectively) (FIG. 15B).

Unstimulated, IO-stimulated, and LPS-stimulated macrophages (CD11b+) were similarly analyzed for the activation markers as above. IO-stimulated macrophages did not significantly up-regulate any of the markers as compared to the unstimulated macrophages (FIG. 15C). However, LPS-stimulated macrophages expressed significantly higher levels of CD86 and CD80/CD86 than unstimulated cells (p values 0.05 and 0.03 respectively) (FIG. 15C).

### IO Inducted Pro-inflammatory Cytokine and Chemokine Production

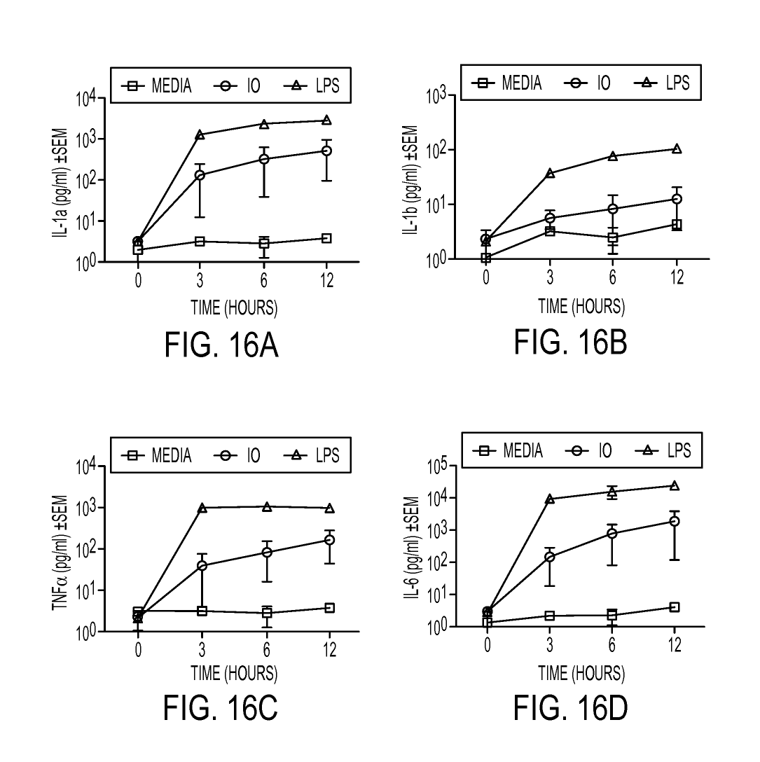

Immature BMDCs were exposed to IO nanoparticles over a 12-hour period and the expression of several cytokines, IL-6, IL-1a, IL-1b, and TNF-α were monitored by RT-PCR. IO nanoparticles significantly increased the production of IL-6, TNF-α, and IL1-b by more than two fold in BMDCs compared to baseline, i.e. 0 hour (FIG. 16). In particular, IL-6 and TNF-α were highly expressed. In general, the cytokine expression profiles of LPS- and IO-stimulated BMDCs were similar.

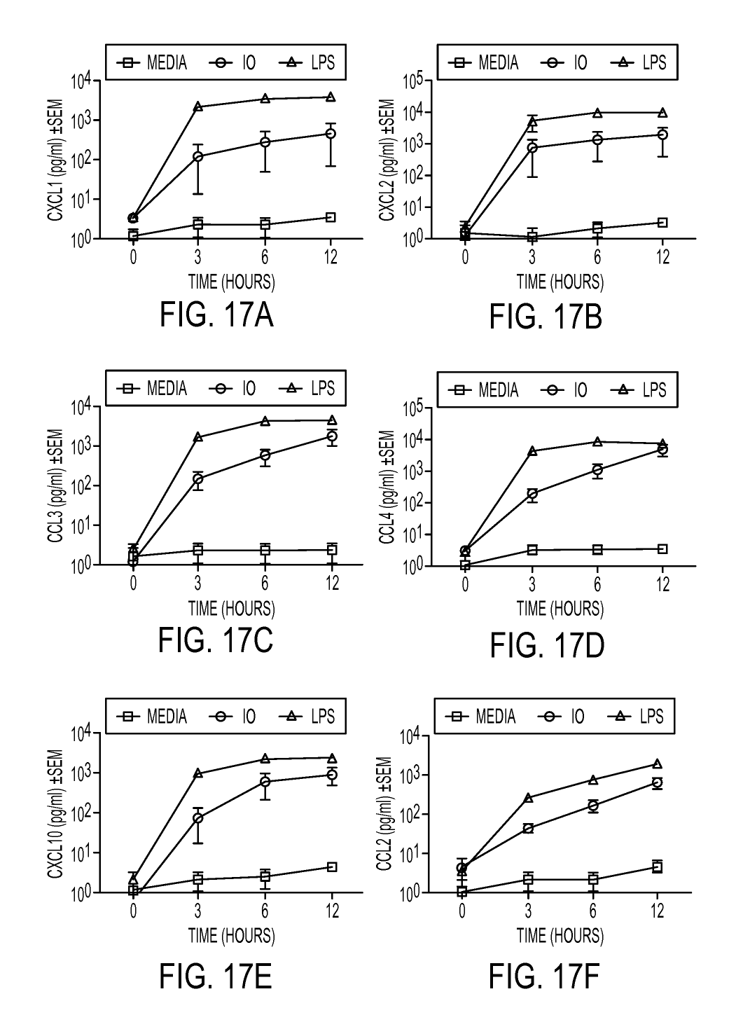

A 32-plex Luminex^{R} assay was performed to test for chemokine production. BMDCs stimulated with either IO nanoparticles or LPS were found to secrete chemokine (FIG. 17) over a 12 hour time course. In comparison to media alone, IO stimulated BMDCs produced higher levels of pro-inflammatory chemokines, including CXCL1, CXCL2, CCL3, CCL4, CXCL10, and CCL2 (FIG. 17). Among them, CCL4 reached the same levels as LPS stimulated BMDCs; and CCL3, CXCL10, and CCL2 reached levels close to those produced by LPS stimulated BMDCs at 12 hours. In general, gradual increases in both cytokine and chemokine levels were observed over time with IO stimulated BMDCs.

### Example 3

Silver, Gold, and CuInS₂ based delivery systems were also tested in various species of animal to determine if they were effective in obtaining immunological responses. The studies were conducted in a manner similar to Examples 1 and 2. Four (4) antigens were tested for antibody production: BSA, human IgG, ovalbumin, and recombinant Plasmodium falciparum mesosporozoite protein (rMSP). The results of the nanoparticle adjuvanted antibody production are summarized in Table 4.

TABLE 4
Antibody production in various animals (covalent conjugated Ag on NM surface)

| Host Animal | Antigen | Nanomaterial | Ab titer (dilution) | | Booster |
|---|---|---|---|---|---|
| SW mice | rMSP (recombinant P falciparum protein) | Quantum dots (8.5 nm) | 0587 | (1:31,250) | 3 |
| SW mice | rMSP (recombinant P falciparum protein) | Iron Oxide (10 nm) | 0.638 | (1:1250) | 3 |
| SW mice | Ovalbumin, 100 uL of 5 mg/mL | CuInS2 (5 nm) | 0.605 | (1:6250) | 3 |
| NZ Rabbit | Ovalbumin, 100 uL of 5 mg/mL | Au (5 nm) | 0.381 | (1:6250) | 3 |
| NZ Rabbit | mIgG (mouse IgG), 100 uL, 5 mg/mL | Iron oxide (10 nm) | 0.338 | (1:640,000) | 3 |
| NZ Rabbit | mIgG (mouse IgG), 100 uL, 5 mg/mL | Quantum dots (8.5 nm) | 0.360 | (1:640,000) | 3 |
| Rabbit | mIgG (mouse IgG), 100 uL, 5 mg/mL | Silver (5 nm) | 0.456 | (1:640,000) | 3 |
| Chicken | BSA (bovine serum albumin), 100 uL, 1 mg/mL | Iron oxide (10 nm) | 0.782 | (1:1000) | 2 |
| Chicken | hIgG (Human IgG), 100 uL, 1 mg/mL | Iron oxide (10 nm) | 2.835 | (1:1000) | 2 |
| Chicken | BSA (bovine serum albumin), 100 uL, 1 mg/mL | Quantum dots (8.5 nm) | 1.273 | (1:1000) | 2 |
| Chicken | hIgG (Human IgG), 100 uL, 1 mg/mL | Quantum dots (8.5 nm) | 2.521 | (1:1000) | 2 |
| Chicken | BSA (bovine serum albumin), 100 uL, 1 mg/mL | Silver (5 nm) | 1.513 | (1:1000) | 2 |
| Chicken | hIgG (Human IgG), 100 uL, 1 mg/mL | Silver (5 nm) | 2.269 | (1:1000) | 2 |

### Example 4

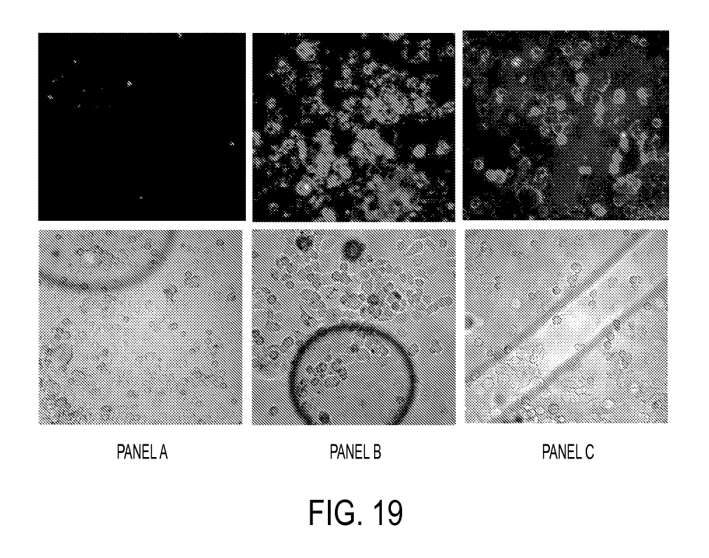

Chicken-hIgG-QD antibodies were tested to see if they would be suitable for detection of human cancer cells. FIG. 19 shows the results of applying the chicken γhIgG-QD antibodies to a plate of cancer cells (SKBR3). The top row of images are pictures of a cell culture taken through a microscope under ultraviolet light. The bottom row of images are pictures of the same cell culture taken under white light. Panel A represents a cell culture exposed to unconjugated QDs. Panel B represents cells exposed to SKBR3+ human/mouse anti-her2+ chicken IgY anti-human IgG-QD. Panel C represents cells exposed to SKBR3+ human/mouse anti-her2+ chicken IgY anti-human IgG-QD. FIG. 18 illustrates that the methods of treatment contemplated by the invention and the vaccines contemplated by the invention exhibit the biological activity that makes them potentially suitable for immunotherapy applications.

### Evaluation of Anti-body Immune Activity

Activity of antibodies generated in the practice of the invention using chickens, rabbits and mice were evaluated using ELISA, fluorescence immunoassay, and parasite growth inhibition. Parasite inhibition using the antibodies against human malaria causing Plasmodium falciparum that were produced using different adjuvants is shown on Table 5. The results indicated that the antibodies produced when the antigens were conjugated with either the iron oxide nanoparticles or quantum dots grown in rabbits had very potent inhibitory effects on the parasites. This is extremely important in considering the applications of adjuvants for disease prevention such as in vaccine delivery or in immunotherapy.

TABLE 5
Immunoactivities of Antibodies against IgG and Ovalbumin

| Host | Nanomat | Antigen | Label | Results |
|---|---|---|---|---|
| Chicken | Iron oxide | hIgG | AP (alkaline | Active |
| | | | phosphatase) | |
| Chicken | Iron oxide | hIgG | HRP (horse raddis | Active |
| | | | peroxidase | |
| Chicken | Iron oxide | hIgG | QD λem 620 nm | Active |
| Rabbit | Iron oxide | ovalbumin | QD λem 620 nm | Active |
| Rabbit | Iron oxide | ovalbumin | Rhodamine B | Active |
| Mouse | Iron oxide | ovalbumin | QD λem 620 nm | Active |
| Mouse | Iron oxide | ovalbumin | Rhodamine B | Active |

### Example 5

### Evaluation of Nanoparticles deposition in Liver, Kidney, Lymph, and Spleen

Rabbit treated with nanoparticles (QD, IO, and Ag) were sacrificed after the nanoparticle mediated delivery of mouse IgG for antibody production. Various organs were collected and inspected for damage. The results shown on FIG. 8 indicated that there was no difference in the organs of the rabbits exposed to the nanoparticles to those of the control. Furthermore, the rabbits did not exhibit any physical distress during the entire duration of the studies. A few of the nanoparticle and control rabbits from each group of treatment were saved and kept for more than 6 months to see if there will be changes in behavior or disease would ensue. The rabbits remained healthy during the entire 6 months incubation period.

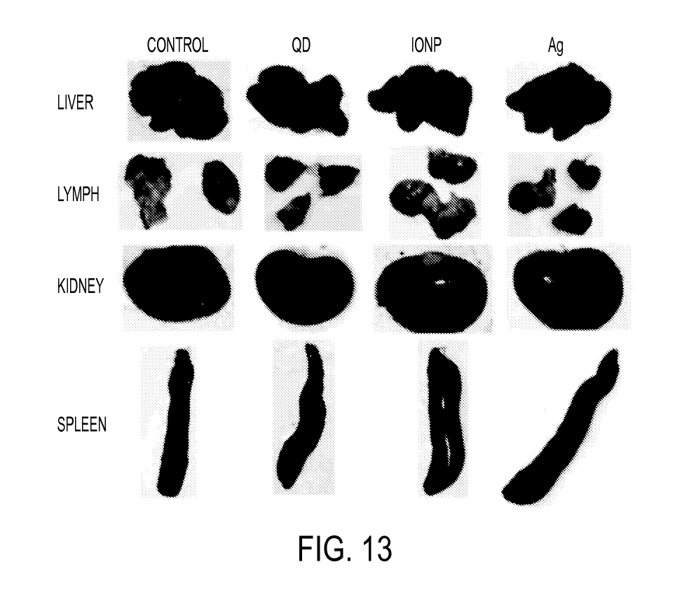

Sections of the organs were homogenized for analysis of nanoparticle deposition. Frozen tissues were sliced and used prepare 5 um tissue sections. These were washed with PBS, followed by incubation with 5% potassium ferrocyanide with 10% hydrochloric acid for 30-45 min. These were examined microscopically for the presence of Fe₂O₃ nanoparticles that form blue coloration resulting from the formation of the iron (II,III) hexacyanoferrate(II,III) (Fe₇(CN)₁₈. Results did not show any iron deposition in any of the organs shown on FIG. 13. This is possibly due to the very low dose at which the IO was used during antigen delivery of mouse IgG.

Tissue preparations from mice that were exposed to CuInS2 nanoparticles were also prepared as above. The tissue preparations were observed under a microscope with UV light source. The results indicated the absence of CuInS2 quantum dots in the various organs.

Toxicity studies showed no Abnormalities in IO Immunized Animals. To demonstrate this, escalating injection doses of IO nanoparticles, up to 4.4 mg per injection, did not cause any abnormalities or changes in the blood chemistries in all four groups of mice, tested after each of the three immunizations. Thus, immunization with IO nanoparticles did not have toxic systemic affects in the animal model.

As many possible embodiments may be made of the invention without departing from the scope thereof, it is to be understood that all matter herein set forth is to be interpreted as illustrative and not in a limiting sense.

While the invention has been described with respect to a various embodiments thereof, it will be understood by those skilled in the art that various changes in detail may be made therein without departing from the spirit, scope, and teaching of the invention. Accordingly, the invention herein disclosed is to be limited only as specified in the following claims.

That which is claimed is:

1. A method of eliciting an immunological response in an animal, said method comprising:
administering a nanostructure to an animal, wherein said nanostructure comprises:
a nanospecies,
a polymer encapsulating said nanospecies, and
an immunogen.

2. A method according to claim 1 wherein said nanostructure does not comprise an adjuvant.

3. A method according to claim 1 wherein the step of administering a nanostructure to an animal occurs in the absence of an adjuvant.

4. A method according to claim 1 wherein said immunogen is attached to said polymer encapsulating said nanospecies.

5. A method according to claim 1 wherein said immunogen is a recombinant protein.

6. A method according to claim 1 wherein the animal is a human.

7. A method according to claim 1 wherein said method is used as a prophylactic vaccination.

8. A method according to claim 1 wherein said immunological response comprises the production of immunoglobulins.

9. A method according to claim 1 wherein said immunological response comprises a T-cell response.

10. A method according to claim 1 wherein said nanostructure is water soluble.

11. A method according to claim 1 wherein said method is used for immunotherapy.

12. A method of vaccinating an animal, said method comprising:
providing a nanostructure wherein said nanostructure comprises
a nanospecies;
a polymer encapsulating said nanospecies; and
an immunogen; and
administering to said animal a quantity of said nanostructure sufficient to initiate an immunological response against said immunogen.

13. A method according to claim 12 wherein the step of administering a nanostructure to said animal occurs in the absence of an adjuvant.

14. A method according to claim 12 wherein said immunological response comprises release of cytokines or chemokines.

15. A method according to claim 12 wherein said immunological response comprises the production of immunoglobulins.

16. A method according to claim 12 wherein said immunogen is a recombinant protein.

17. A method according to claim 12 wherein said nanospecies is selected from the group consisting of quantum dots, a metallic nanoparticles, and metal oxide nanoparticles.

18. A method according to claim 12 wherein said animal is a human.

19. A vaccine for vaccinating an animal against a pathogen, said vaccine comprising:
a nanostructure composition, said composition comprising
a nanospecies;
a polymer encapsulating said nanospecies; and
an immunogen; and
wherein said nanostructure does not comprise an adjuvant.

20. A vaccine according to claim 19 wherein said immunogen is a recombinant protein.

21. A vaccine according to claim 19 wherein said nanospecies is selected from the group consisting of quantum dots, a metallic nanoparticles, and metal oxide nanoparticles.

22. A vaccine according to claim 19 wherein said animal is a human.

23. A vaccine according to claim 19 wherein said immunological response comprises release of cytokines or chemokines.

24. A vaccine according to claim 19 wherein said immunological response comprises the production of immunoglobulins.

25. A vaccine according to claim 19 that is administered prophylactically.

26. A vaccine according to claim 19 that is administered before or after exposure to said pathogen.

27. A vaccine according to claim 19 wherein said pathogen is a cancer cell.
